(19) United States
(12) Patent Application Publication
(10) Pub. No.: US 2025/0318997 A1
(43) Pub. Date: Oct. 16, 2025

(54) COMPOSITIONS AND METHODS FOR PREVENTING DENTAL CARIES

(71) Applicant: THE TRUSTEES OF THE UNIVERSITY OF PENNSYLVANIA, Philadelphia, PA (US)

(72) Inventors: Hyun KOO, Philadelphia, PA (US); David CORMODE, Philadelphia, PA (US); Yue HUANG, Frederick, MD (US); Nil K. PANDEY, Philadelphia, PA (US)

(73) Assignee: THE TRUSTEES OF THE UNIVERSITY OF PENNSYLVANIA, Phiadelphia, PA (US)

(21) Appl. No.: 19/174,250

(22) Filed: Apr. 9, 2025

Related U.S. Application Data
(63) Continuation of application No. PCT/US2023/034093, filed on Sep. 29, 2023.
(60) Provisional application No. 63/414,757, filed on Oct. 10, 2022.

(51) Int. Cl. A61K 6/20; A61K 8/02; A61K 8/19; A61K 8/362; A61K 8/73; A61Q 11/00; B82Y 5/00

(52) U.S. Cl. CPC A61K 6/20; A61K 8/0245; A61K 8/19; A61K 8/362; A61K 8/73; A61Q 11/00; B82Y 5/00

(57) ABSTRACT

The presently disclosed subject matter relates to compositions and methods for preventing and/or treating of an oral disease and/or a biofilm-associated disease. In particular, the presently disclosed subject matter provides a composition for preventing and/or treating of an oral disease comprising (a) one or more iron oxide nanoparticles and (b) stannous fluoride (SnF₂) that provide synergistic properties, including enhanced solubility, stability, co-delivery and catalytic activity, while creating a protective antibacterial and anti-demineralization layer on the target surface for disease prevention.

### CROSS-REFERENCE TO RELATED APPLICATION

This application is a continuation of International Patent Application No. PCT/US2023/034093, filed Sep. 29, 2023, which claims priority to U.S. Provisional Patent Application Ser. No. 63/414,757, filed Oct. 10, 2022, which are hereby incorporated by reference herein in their entireties.

### GRANT INFORMATION

This invention was made with government support under DE025848 awarded by the National Institutes of Health. The government has certain rights in the invention.

### BACKGROUND

Dental caries can be a prevalent and costly biofilm-induced disease that causes the destruction of the mineralized tooth tissue. Despite advances, at the time of filing it affects 3.1 billion people worldwide, with costs exceeding US $290 billion. In caries-inducing (cariogenic) biofilms, microorganisms form highly protected biostructures that create localized acidic pH microenvironments, promoting cariogenic bacteria growth and acid dissolution of tooth-enamel.

Certain antimicrobials can be insufficient to prevent dental caries in high-risk individuals where pathogenic dental biofilms rapidly accumulate under sugar-rich diets and poor oral hygiene that enables firm bacterial adhesion to teeth and rampant enamel acid demineralization leading to cavitation. Conversely, fluoride is the mainstay for caries treatment by reducing enamel demineralization. However, it is ineffective against biofilms and does not offer complete protection against dental caries. Hence, the high prevalence of dental caries continues both in the US and worldwide.

Accordingly, there exists a need for compositions and methods for preventing and/or treating dental caries.

### SUMMARY

The presently disclosed subject matter provides compositions and methods for preventing and/or treating an oral disease and/or a biofilm-associated disease.

In certain embodiments, the disclosed subject matter provides a composition for preventing and/or treating of an oral disease comprising (a) one or more iron oxide nanoparticles and (b) stannous fluoride (SnF₂). That are synergistic in disrupting biofilms and preventing dental caries (SnF₂ enhances catalytic antibiofilm/antimicrobial activity of iron oxide NP whereas NP stabilizes and deliver SnF₂ into biofilm and onto enamel while creating a protective layer) In certain embodiments, the oral disease can include dental caries. In non-limiting embodiments, the one or more iron oxide nanoparticles can include nanoparticles having a diameter of about 1 nm to about 1000 nm. In non-limiting embodiments, the composition can further include fluoride, copper, calcium phosphate, or a combination thereof.

In certain embodiments, the one or more iron oxide nanoparticles can include nanoparticles that have a polymeric coating. In non-limiting embodiments, the polymeric coating can include a biopolymer, dextran, chitosan, citrate, or combinations thereof. In non-limiting embodiments, Sn²⁺ of the SnF₂ is bound by carboxylate groups in a carboxymethyl-dextran coating of the one or more iron oxide nanoparticles. In certain embodiments, the one or more iron oxide nanoparticles comprise nanoparticles that do not have a polymeric coating.

In certain embodiments, the composition can include an active agent. In non-limiting embodiments, the active agent can include an antimicrobial, an antibiotic, or a combination thereof.

In certain embodiments, the composition can be formulated as a solution, a cream, a gel, a paste, a paste, a strip, a lozenge, a gum, a gummy, a gummy bear, a resin, a sealant, a coating or a combination thereof. In non-limiting embodiments, the composition can be formulated as an aqueous solution.

In certain embodiments, a concentration of the one or more Fer nanoparticles can be from about 1 mg/ml to about 5 mg/ml. In non-limiting embodiments, a concentration of the SnF₂ ranges from about 1 ppm of F to about 1000 ppm of F.

The disclosed subject matter provides a method for preventing and/or treating a biofilm-associated disease. The method can include administering to a subject an effective amount of a composition comprising one or more iron oxide nanoparticles and stannous fluoride (SnF₂).

In certain embodiments, the method can further include incubating the one or more iron oxide nanoparticles and SnF₂ for a predetermined time. In non-limiting embodiments, the predetermined time ranges from about 1 minute to about 12 months.

In certain embodiments, the method can further include performing a polymeric coating on the one or more iron oxide nanoparticles. In non-limiting embodiments, the polymeric coating comprises a biopolymer, dextran, carboxymethyl dextran, chitosan, citrate, or combinations thereof.

In certain embodiments, the method can further include creating a protective layer at a target surface using the composition. In non-limiting embodiments, the protective layer can be an antibacterial layer that can be enriched with Sn, iron, and fluoride for preventing enamel demineralization.

It can also serve as delivery system for SnF₂ into biofilm and onto enamel surface.

In certain embodiments, the biofilm can be generated by a biofilm-forming microbe. In non-limiting embodiments, the biofilm-forming microbe can include S. mutans, P. aeruginosas, E. coli, E. faecalis, B. subtilis, S. aureus, Vibrio cholerae, Candida albicans, or a combination thereof. In non-limiting embodiments, the biofilm can be present on a surface of a tooth, an industrial material, a naval material, skin, mucosal/soft tissue, an interior of a tooth (endodontic canal), lung (cystic fibrosis), urinary tract, or a medical device.

In certain embodiments, the disclosed composition can further include citric acid. In non-limiting embodiments, the iron oxide nanoparticles can be coated with the citric acid. In non-limiting embodiments, the citric acid can range from about 1 μg/ml to about 1000 μg/ml.

The disclosed subject matter will be further described below, with reference to example embodiments shown in the drawings.

### BRIEF DESCRIPTION OF THE DRAWINGS

FIGS. 1A-1E depict the effect of different ratios of Fer (1 mg of Fe/ml) and SnF₂ (0-250 ppm of F) on (FIG. 1A) the bacterial viability and (FIG. 1B) the mass of biofilm. FIGS. 1C-1D depict the effect of different ratios of Fer (0-1 mg of Fe/ml) and SnF₂ (250 ppm of F) on (FIG. 1C) the bacterial viability and (FIG. 1D) the mass of biofilm. FIG. 1E depicts confocal microscopy images (right) and quantification (left) of biofilm treatment with Fer (1 mg of Fe/ml) and SnF₂ (250 ppm of F).

FIG. 2A-2G illustrate photographs, NMR spectra, and UV-vis absorption spectra for various compositions. FIG. 2A provides photographs of Fer and SnF₂ at different concentrations at pH 4.5 and 5.5 after 24 h incubation. FIG. 2B provides photographs of carboxymethyl-dextran (CMD) and SnF₂ at pH 5.5 before or after 24 h incubation. FIG. 2C depicts ¹H NMR spectra of CMD and CMD+SnF₂. FIG. 2D provides photographs of SnF₂ in different conditions at pH 4.5 (0.1 M sodium acetate buffer). The samples are: 1. SnF₂ alone, 2. SnF₂+CMD, 3. SnF₂+dextran, 4. SnF₂+citric acid, 5. SnF₂+L-ascorbic acid, and 6. SnF₂+poly(acrylic acid). FIG. 2E provides UV-vis absorption spectra of SnF₂ (250 ppm of F) with or without CMD (1 mg/ml) at pH 4.5 (0.1 M sodium acetate buffer) after 0 or 24 h incubation. FIG. 2F provides UV-vis absorption spectra of SnF₂ (250 ppm of F) with or without dextran (1 mg/ml) at pH 4.5 (0.1 M sodium acetate buffer) after 0 or 24 h incubation. FIG. 2G provides photographs of SnF₂ with various amounts of mannitol at pH 4.5 (0.1 M sodium acetate buffer) after 0 or 24 h incubation. The samples are: 1. SnF₂ alone, 2. SnF₂+1 mg/ml mannitol, 3. SnF₂+2 mg/ml mannitol, and 4. SnF₂+10 mg/ml mannitol.

FIG. 3A-3F depict the effects of SnF₂, Fer, or Fer+SnF₂ on absorbance and intensity. FIG. 3A depicts the change in the absorption of TMB (chromogenic substrate) at 652 nm in different conditions. FIG. 3B depicts UV-vis absorption spectra of TMB in the presence of SnF₂, Fer, or Fer+SnF₂ at the times indicated. FIG. 3C depicts the peroxidase-like activity of Fer and Fer+SnF₂ at three pH values (4.5, 5.5, and 6.5) as determined by the colorimetric assay using TMB. FIG. 3D depicts the change in the absorption of OPD at 450 nm in different conditions. FIG. 3E depicts a comparison of the change in PL intensities of DCF at 520 nm at various conditions. FIG. 3F depicts the change in PL intensity of 7-hydroxycoumarin at 452 nm as a function of time in the presence of Fer with or without SnF₂.

FIGS. 4A-4F depict the effect of various compounds on the catalytic activity of Fe from various sources of iron. FIGS. 4A-4C depict the effect of (FIG. 4A) NaF (20 μg/ml), (FIG. 4B) BaF₂ (20 or 30 μg/ml), and (FIG. 4C) SnCl₂ (20 μg/ml) on the catalytic activity of Fer (20 μg of Fe/ml) in 0.1 M sodium acetate buffer (pH 4.5). FIG. 4D depicts amounts of iron in the nanoparticle pellet and filtrate at pH 4.5 via ICP-OES. FIG. 4E depicts a comparison of the catalytic activity of the released iron ions and nanoparticle pellet in different conditions at pH 4.5 as measured by TMB assay. FIG. 4F depicts a comparison of the catalytic activity of leached iron ions at three pH values (4.5, 5.5, and 6.5).

FIGS. 5A-5D depict caries scores and histology of the biofilm-associated oral disease in vivo. FIG. 5A-5C depict caries score in initial lesions (5A), moderate lesions (5B), and severe lesions (5C). FIG. 5D shows histological analysis of gingival tissue.

FIGS. 6A-6D depict the effects of Fer and SnF₂ on the oral microbiome in vivo after treatment. FIG. 6A shows the relative abundance of oral microbiota. FIGS. 6B-6C show the comparative diversity by Richness (6B) and Shannon (6C). FIG. 6D illustrates principle coordinate analysis of various treatment groups.

FIG. 7 provides enamel surface analysis, showing a protective layer of Sn, iron, and fluoride.

FIG. 8 provides a diagram showing the interactions and therapeutic activity of the combined treatment of Fer and SnF₂, showing that nanoparticles can also deliver SnF₂ to the target region, e.g. into biofilm and onto enamel surface.

FIGS. 9A-9D depict bacterial viability and mass of biofilm for various treatments. FIG. 9A depicts the bacterial viability after treatment with NaF or SnF₂ at 1000 ppm of F. FIG. 9B depicts the mass of biofilm after treatment with NaF or SnF₂ at 1000 ppm of F. FIG. 9C depicts the bacterial viability after treatment with Fer+NaF or Fer+SnF₂ at 1 mg of Fe/ml, 1000 ppm of F, and 1% of H₂O₂. FIG. 9D depicts the mass of biofilm after treatment with Fer+NaF or Fer+SnF₂ at 1 mg of Fe/ml, 1000 ppm of F, and 1% of H₂O₂.

FIG. 10 depicts example TEM images of Fer and Fer+SnF₂ after 1 h incubation in 0.1 M sodium acetate buffer (pH 4.5).

FIGS. 11A-11B depict UV-visible absorption spectra of SnF₂ (250 ppm of F) in 0.1 M sodium acetate buffer (pH 4.5) with or without (11A) citric acid (1 mg/ml) and (11B) L-ascorbic acid (1 mg/ml) at the time points indicated.

FIG. 12 depicts the effects of DMSO on the catalytic activity of Fer (20 μg of Fe/ml)+SnF₂ (20 μg/ml) in 0.1 M sodium acetate buffer (pH 4.5).

FIGS. 13A-13B depict the effect of incubation time on the catalytic activity of Fer (20 μg of Fe/ml) with or without SnF₂ (20 μg/ml) in 0.1 M sodium acetate buffer (pH 4.5) with incubation in 10 minute intervals (13A) and 1 hour intervals (13B).

FIGS. 14A-14B depict the effect of various compositions on decolorization and UV-vis absorption spectra. FIG. 14A depicts a comparison of the decolorization efficiency of Fer+H₂O₂ with or without SnF₂. FIG. 14B depicts example UV-vis absorption spectra of methylene blue in the presence of Fer+H₂O₂ with or without SnF₂.

FIG. 15 depicts the change in PL intensity of 7-hydroxycoumarin at 452 nm as a function of time with or without SnF₂ (20 μg/ml).

FIG. 16 depicts the amount of iron in the filtrate of the combination of Fer (0.5 mg of Fe/ml) and SnF₂ (0.5 mg/ml) after 1 h incubation at three different pH values via ICP-OES.

FIGS. 17A-17B depict the bacterial viability (17A) and biofilm mass (17B) with the varied concentration of Fer (0-1 mg of Fe/ml) and SnF₂ (0-250 ppm of F).

FIG. 18 depicts a comparison of the catalytic activity of carboxymethyl-dextran (CMD)-coated IONP (Fer formulation contains CMD; CMD is a coating agent in Fer) with or without SnF₂. The presence of SnF₂ increases the catalytic activity.

FIG. 19 depicts a comparison of the catalytic activity of citric acid-coated IONP with or without SnF₂.

FIG. 20 depicts an effect of post-mixed SnF₂ on the catalytic activity of Fer.

FIG. 21 provides photographs of Fer+SnF₂ mixed with various amounts of citric acid.

FIG. 22 depicts an evaluation of catalytic activity of Fer/SnF₂ formulation in the presence of various amounts of citric acid. The catalytic activity is further enhanced in the presence of citric acid.

FIG. 23 depicts the mixing of Fer/SnF₂ with hydrogen peroxide using a dual barrel syringe. The left barrel contains Fer+SnF₂, while the right barrel contains H₂O₂.

The accompanying drawings, which are incorporated and constitute part of this disclosure, illustrate certain embodiments and serve to explain the principles of the disclosed subject matter.

### DETAILED DESCRIPTION

The presently disclosed subject matter provides compositions and formulations thereof for the treatment of oral diseases as well as for industrial and other medical applications. The presently disclosed subject matter further provides methods of using the compositions and formulations of the present disclosure in the elimination of biofilms, the prevention of biofilm formation, matrix degradation and/or the inhibition of microorganism viability and growth within the biofilm as well as protection of dental enamel against demineralization.

As used herein, a “biofilm” includes an extracellular matrix and one or more microorganisms such as, but not limited to, bacteria, fungi, algae and protozoa, which are attached to a surface. For example, but not by way of limitation, such surfaces can include tooth, mucosal, apatitic, bone and abiotic (e.g., implant, dentures, pipes, etc.) surfaces. Biofilms can form on living or non-living surfaces and can exist in natural and industrial settings.

Biofilms that can be prevented, eliminated and/or treated by the compositions and/or formulations of the present disclosure include, but are not limited to, biofilms present within the oral cavity, e.g., on the surface of teeth, on the surface of mucosal/soft-tissues such as gingivae/periodontium and inside a tooth canal (e.g., endodontic canal). In certain embodiments, biofilms that can be prevented, eliminated and/or treated by the compositions and/or formulations of the present disclosure include biofilms on the urinary tract, lung, gastrointestinal tract, on and/or within chronic wounds, and present on the surface (e.g., implants) and within medical devices and medical lines, e.g., catheters, medical instruments and medical tubing. Additional non-limiting examples of biofilms include biofilms present within industrial equipment and materials, e.g., pipes for water, sewage, oil or other substances. In certain embodiments, compositions and/or formulations of the present disclosure can be used to treat or clean the hulls of ships and other naval craft.

As used herein, the term “about” or “approximately” means within an acceptable error range for the particular value as determined by one of ordinary skill in the art, which will depend in part on how the value is measured or determined, i.e., the limitations of the measurement system. For example, “about” can mean a range of up to 20%, up to 10%, up to 5%, and/or up to 1% of a given value.

As noted above, the compositions of the present disclosure can be used to reduce the growth and/or inhibit the viability of one or more microorganisms, e.g., microbes in a biofilm. For example, and not by way of limitation, the microbes can include Streptococcus mutans (S. mutans), Streptococcus sobrinus, Streptococcus sanguis (sanguinis), Streptococcus gordonii, Streptococcus oralis, Streptococcus mitis, Actinomyces odontolyticus, Actinomyces viscosus, Aggregatibacter actinomycetemcomitans, Lactobacillus spp., Porphyromonas gingivalis, Prevotella intermedia, Bacteroides forsythus, Treponema denticola, Fusobacterium nucleatum, Campylobacter rectus, Eikenella corrodens, Veillonella spp., Micromonas micros, Porphyromonas cangingivalis, Haemophilus actinomycetemcomitans Actinomyces spp., Bacillus spp., Mycobacterium spp., Fusobacterium spp., Streptococcus spp., Staphylococcus aureus, Streptococcus pyogenes, Streptococcus agalectiae, Proteus mirabilis, Klebsiella pneumoniae, Acinetobacter spp., Enterococcus spp., Prevotella spp., Porphyromonas spp., Clostridium spp., Stenotrophomonas maltophilia, P. cangingivalis, Candida albicans, Escherichia coli and Pseudomonas aeruginosa. In certain embodiments, the bacteria are S. mutans, which are present within biofilms found in the oral cavity, e.g., on the surface of teeth.

The presently disclosed subject matter provides compositions that include one or more iron oxide nanoparticles and stannous fluoride (SnF₂). The disclosed compositions can be used for treating or preventing dental caries. In non-limiting embodiments, the disclosed compositions can also be used for the treatment and/or elimination of biofilms and/or the prevention of biofilm formation. For example, and not by way of limitation, compositions disclosed herein can be used to treat existing biofilms, e.g., biofilms already present on a surface. In certain embodiments, compositions of the present disclosure can be used to prevent the initiation and/or formation of biofilms, e.g., by coating a surface with a disclosed composition. As disclosed herein, the disclosed compositions of the present disclosure can bind to target surfaces (e.g., tooth surfaces) as well as penetrate and be retained within a biofilm to disrupt the extracellular matrix of the biofilm and reduce the growth and/or kill the bacteria embedded within the biofilm. In non-limiting embodiments, the disclosed nanoparticles can deliver SnF₂ to the target biofilm and/or the enamel surface. The treatment of the disclosed compositions can create a protective outer layer in the enamel enriched with Sn, iron, and fluoride that can protect against enamel demineralization while also serving as a reservoir for Sn, which can serve as an antibacterial layer right at the tooth surface.

In certain embodiments, the disclosed nanoparticles can include nanoparticles made from iron oxide. In non-limiting embodiments, the disclosed nanoparticles can include one or more ferumoxytol (Fer) nanoparticles. The disclosed nanoparticles can be used against cariogenic biofilms when used topically through selective pathogen binding and acidic pH-activation of hydrogen peroxide via catalytic (peroxidase-like) activity.

In certain embodiments, the composition can have a nanoparticle concentration of about 0.01 to about 10.0 mg/ml. For example, and not by way of limitation, composition can have an Fer nanoparticles concentration from about 0.01 to about 9.0 mg/ml, from about 0.01 to about 8.0 mg/ml from about 0.01 to about 7.0 mg/ml, from about 0.01 to about 6.0 mg/ml, from about 0.01 to about 5.0 mg/ml, from about 0.01 to about 4.0 mg/ml, from about 0.01 to about 3.0 mg/ml, from about 1.0 to about 2.0 mg/ml, from about 2.0 to about 10.0 mg/ml, from about 3.0 to about 10.0 mg/ml, from about 4.0 to about 10.0 mg/ml, from about 5.0 to about 10.0 mg/ml, from about 6.0 to about 10.0 mg/ml, from about 7.0 to about 10.0 mg/ml, from about 8.0 to about 10.0 mg/ml or from about 9.0 to about 10.0 mg/ml. In certain embodiments, the Fer nanoparticles can have an iron concentration of about 1 mg/ml.

In certain embodiments, the nanoparticles can have a hydrodynamic diameter from about 1 nm to about 1000 nm. For example, and not by way of limitation, the nanoparticles can have a hydrodynamic diameter from about 10 nm to about 100 nm, from about 15 nm to about 100 nm, from about 20 nm to about 100 nm, from about 25 nm to about 100 nm, from about 30 nm to about 100 nm, from about 35 nm to about 100 nm, from about 40 nm to about 100 nm, from about 45 nm to about 100 nm, from about 50 nm to about 100 nm, from about 55 nm to about 100 nm, from about 60 nm to about 100 nm, from about 65 nm to about 100 nm, from about 70 nm to about 100 nm, from about 75 nm to about 100 nm, from about 80 nm to about 100 nm, from about 85 nm to about 100 nm, from about 90 nm to about 100 nm, from about 95 nm to about 100 nm, from about 5 nm to about 95 nm, from about 5 nm to about 90 nm, from about 5 nm to about 85 nm, from about 5 nm to about 80 nm, from about 5 nm to about 75 nm, from about 5 nm to about 70 nm, from about 5 nm to about 65 nm, from about 5 nm to about 60 nm, from about 5 nm to about 55 nm, from about 5 nm to about 50 nm, from about 5 nm to about 45 nm, from about 5 nm to about 40 nm, from about 5 nm to about 35 nm, from about 5 nm to about 30 nm, from about 5 nm to about 25 nm, from about 5 nm to about 20 nm, from about 5 nm to about 15 nm or from about 5 nm to about 10 nm. In certain embodiments, the nanoparticles can have a hydrodynamic diameter from about 10 nm to about 25 nm.

In certain embodiments, the disclosed composition can include fluoride (e.g., stannous fluoride). In certain embodiments, fluoride can be present within a formulation of the present disclosure at a concentration of about 10 parts per million (ppm) of F to about 5,000 ppm, e.g., from about 100 ppm to about 4,500 ppm, from about 100 ppm to about 4,000 ppm, from about 100 ppm to about 3,500 ppm, from about 100 ppm to about 3,000 ppm, from about 100 ppm to about 2,500 ppm, from about 100 ppm to about 2,000 ppm, from about 100 ppm to about 1,500 ppm, from about 100 ppm to about 1,000 ppm, from about 100 ppm to about 500 ppm or from about 200 ppm to about 400 ppm. In certain embodiments, fluoride is present at a concentration from about 200 ppm to about 300 ppm, e.g., about 250 ppm. In certain embodiments, fluoride is present at a concentration between about 1 ppm to about 1000 ppm.

In certain embodiments, the Fer nanoparticles can include a polymeric coating, for example, and not by way of limitation, the polymeric coating can include chitosan, citrate, poly(acrylic acid) or dextran. In certain embodiments, the polymeric coating can be dextran or a modified dextran. For example, and not by way of limitation, the dextran can be cross-linked, aminated, carboxylated or modified with diethyl aminoethyl moieties. In certain embodiments, the dextran used in the coating of Fer nanoparticles of the present disclosure can have a molecular weight from about 1 kDa to about 100 kDa, e.g., from about 1 kDa to about 90 kDa, from about 1 kDa to about 80 kDa, from about 1 kDa to about 70 kDa, from about 1 kDa to about 60 kDa, from about 1 kDa to about 50 kDa, from about 1 kDa to about 40 kDa, from about 1 kDa to about 30 kDa, from about 1 kDa to about 20 kDa, from about 1 kDa to about 10 kDa, from about 1 kDa to about 5 kDa, from about 5 kDa to about 100 kDa, from about 10 kDa to about 100 kDa, from about 20 kDa to about 100 kDa, from about 30 kDa to about 100 kDa, from about 40 kDa to about 100 kDa, from about 50 kDa to about 100 kDa, from about 60 kDa to about 100 kDa, from about 70 kDa to about 100 kDa, from about 80 kDa to about 100 kDa or from about 90 kDa to about 100 kDa. In non-limiting embodiments, the disclosed composition can include nanoparticles without a polymeric coating.

In certain embodiments, the disclosed composition includes one or more iron oxide nanoparticles and stannous fluoride (SnF₂). In non-limiting embodiments, the nanoparticles can be used to deliver SnF₂ to the target biofilm and/or the enamel surface. Sn²⁺ of the SnF₂ can be bound by carboxylate groups in a carboxymethyl-dextran coating of one or more Fer nanoparticles making stannous fluoride soluble in aqueous solution. For example, Fer can stabilize SnF₂ through Sn²⁺ interactions with the carboxylate group in the carboxymethyl-dextran coating of the nanoparticles in aqueous solution. The inclusion of SnF₂ can enhance the catalytic (peroxidase-like) activity of Fer under pathological conditions (acidic pH) but not at physiological pH (pH>6.5), thereby increasing its specificity and antibiofilm activity in cariogenic conditions. In non-limiting embodiments, the disclosed compound can keep two oxidation states (e.g., Sn²⁺, Sn⁴⁺). For example, Sn can keep Sn²⁺ rather than Sn⁴⁺ when exposed to oxidizing agents (e.g., H₂O₂ or OH).

In certain embodiments, the nanoparticles mixed with SnF₂ can have a hydrodynamic diameter from about 1 nm to about 1000 nm. For example, and not by way of limitation, the Fer nanoparticles mixed with SnF₂ can have a hydrodynamic diameter from about 10 nm to about 100 nm, from about 15 nm to about 100 nm, from about 20 nm to about 100 nm, from about 25 nm to about 100 nm, from about 30 nm to about 100 nm, from about 35 nm to about 100 nm, from about 40 nm to about 100 nm, from about 45 nm to about 100 nm, from about 50 nm to about 100 nm, from about 55 nm to about 100 nm, from about 60 nm to about 100 nm, from about 65 nm to about 100 nm, from about 70 nm to about 100 nm, from about 75 nm to about 100 nm, from about 80 nm to about 100 nm, from about 85 nm to about 100 nm, from about 90 nm to about 100 nm, from about 95 nm to about 100 nm, from about 5 nm to about 95 nm, from about 5 nm to about 90 nm, from about 5 nm to about 85 nm, from about 5 nm to about 80 nm, from about 5 nm to about 75 nm, from about 5 nm to about 70 nm, from about 5 nm to about 65 nm, from about 5 nm to about 60 nm, from about 5 nm to about 55 nm, from about 5 nm to about 50 nm, from about 5 nm to about 45 nm, from about 5 nm to about 40 nm, from about 5 nm to about 35 nm, from about 5 nm to about 30 nm, from about 5 nm to about 25 nm, from about 5 nm to about 20 nm, from about 5 nm to about 15 nm or from about 5 nm to about 10 nm. In certain embodiments, the Fer nanoparticles mixed with SnF₂ can have a hydrodynamic diameter from about 10 nm to about 25 nm.

In certain embodiments, the composition can include H₂O₂ at a concentration of about 0.01% to about 3.0% v/v. In certain embodiments, the composition can include H₂O₂ at a concentration of about 0.05% to about 3.0%, about 0.1% to about 0.25%, about 0.1% to about 0.5%, about 0.1% to about 0.75%, about 0.1% to about 1.0%, about 0.1% to about 1.5%, about 0.1% to about 1.75%, about 0.1% to about 2.0%, about 0.1% to about 2.25%, about 0.1% to about 2.5% or about 0.1% to about 2.75%. In certain embodiments, the one or more nanoparticles can catalyze H₂O₂ to form one or more free radicals that can degrade and/or digest the extracellular matrix of the biofilm and/or kill bacteria. For example, and not by way of limitation, the one or more types of free radicals can degrade the extracellular matrix of the biofilm and kill bacteria simultaneously. In certain embodiments, the nanoparticles can catalyze H₂O₂ to produce free radicals, for example, and not by way of limitation, hydroxyl radicals (·OH).

In certain embodiments, a composition of the present disclosure can include a mixture of nanoparticles that have different polymeric coatings, e.g., one or more types of nanoparticles within the composition can have a dextran coating, and one or more types of nanoparticles within the composition can have a modified dextran coating.

The presently disclosed subject matter further provides formulations that incorporate the disclosed nanoparticle compositions, e.g., a composition that includes one or more nanoparticles with SnF₂ and/or a composition that includes one or more nanoparticles with SnF₂ and H₂O₂. For example, and not by way of limitation, the formulations can include oral care products and products for delivering the composition into the oral cavity and commercial products for the delivery of the composition into a medical device, a naval material and/or vessel or industrial material. In certain embodiments, the compositions can be incorporated into materials for use in manufacturing medical devices, e.g., medical tubing and catheters, for use in manufacturing oral prosthetics, e.g., dentures and implants, and for use in manufacturing industrial materials, e.g., pipes or ship hulls. In certain embodiments, formulations of the present disclosure can be applied topically, e.g., applied to chronic wounds or skin diseases as treatment. In certain embodiments, formulations of the present disclosure can be used as a spray and/or paint to coat one or more surfaces of an industrial material or a ship hull.

In certain embodiments, the disclosed composition can be formulated as a solution, a cream, a gel, a paste, a paste, a strip, a lozenge, a gum, a gummy, a gummy bear, a resin, a sealant, a coating or a combination thereof. For example, the composition can be formulated as a gummy bear containing xylitol. In non-limiting embodiments, the disclosed composition can be formulated as an aqueous solution.

In certain embodiments, the disclosed compositions of the present disclosure can be incorporated into a formulation for the delivery of the composition into a medical device or industrial material. For example, the composition can be incorporated into a liquid formulation, as disclosed above. In certain embodiments, the composition can be incorporated into a lubricant, ointment, cream or gel that includes a diluent (e.g., Tris, citrate, acetate or phosphate buffers) having various pH values and ionic strengths, solubilizers such as TWEEN™ or Polysorbate, preservatives such as thimerosal, parabens, benzylalconium chloride or benzyl alcohol, antioxidants such as ascorbic acid or sodium metabisulfite and other components such as lysine or glycine. Alternatively or additionally, catheter or medical tubing materials can be impregnated with the disclosed composition of the present disclosure to prevent the formation of biofilms on the surface of and/or within the catheter or tubing.

In certain embodiments, the disclosed subject matter can further include additional compounds. For example, additional compounds can include fluoride, copper, calcium phosphate, xylitol or a combination thereof. In non-limiting embodiments, the disclosed composition can further include an active agent (e.g., antimicrobials and antibiotics). For example, the active agent can include chlorhexidine, fluconazole, nystatin, essential oils, antimicrobial peptides, CPC, triclosan, quaternary salts, small molecules, flavonoids, terpenoids, alkaloids, enzymes, lectins or combinations thereof.

In certain embodiments, the disclosed composition can further include an active agent (e.g., antimicrobials and antibiotics). For example, the active agent can include chlorhexidine, fluconazole, nystatin, essential oils, antimicrobial peptides, CPC (Cetylpyridinium chloride), triclosan, quaternary salts, small molecules, flavonoids, terpenoids, alkaloids, enzymes, lectins or combinations thereof.

The presently disclosed subject matter further provides methods for using the disclosed compositions and/or formulations. The methods of the present disclosure can be used to treat and/or prevent biofilms and/or biofilm-related infections. For example, and not by way of limitation, administration of a composition or formulation of the present disclosure can be used to inhibit the formation of biofilms, inhibit further accumulation of biofilm, promote the disruption or disassembly of existing biofilms and/or weaken an existing biofilm. For example, but not by way of limitation, the compositions and/or formulations of the present disclosure can be used to treat biofilms that promote oral disease. Oral diseases can include, but are not limited to, diseases and disorders that affect the oral cavity or associated medical conditions. For example, oral diseases include, but are not limited to, dental caries, as well as periodontal diseases such as gingivitis, adult periodontitis, early-onset periodontitis, peri-implantitis and endodontic infections.

In certain embodiments, a composition or formulation of the present disclosure can be used to treat and/or prevent biofilm-associated mucosal infections including, for example, denture stomatitis, mucositis and oral candidiasis. In certain embodiments, methods of the disclosed subject matter can be used to treat and/or prevent diseases or disorders including, but not limited to, urinary tract infections, catheter infections, middle-ear infections, wounds and infections of implanted medical devices, e.g., artificial joints and artificial valves.

As used herein, “treatment” (and grammatical variations thereof such as “treat” or “treating”) refers to clinical intervention in an attempt to alter the natural course of the individual being treated, and can be performed either for prophylaxis or during the course of the disease. Desirable effects of treatment include, but are not limited to, preventing the occurrence or recurrence of the disease, alleviation of symptoms, diminishment of any direct or indirect pathological consequences of the disease, and decreasing the rate of disease progression or amelioration of the disease state. In certain embodiments, the compositions and formulations of the present disclosure can be used to delay the development of a disease or to slow the progression of a disease. In certain embodiments, treatment can refer to the elimination, removal and/or reduction of existing biofilms. In certain embodiments, prevention can refer to impeding the initiation or formation of a biofilm on a surface.

An “individual,” “patient,” or “subject,” as used interchangeably herein, refers to a mammal. Mammals include, but are not limited to, domesticated animals (e.g., cows, sheep, cats, dogs and horses), primates (e.g., humans and non-human primates such as monkeys), rabbits and rodents (e.g., mice and rats). In certain embodiments, the individual or subject is a human.

In certain embodiments, methods for the prevention and treatment of oral disease and/or for the prevention and treatment of biofilms in a subject can include administering an effective amount of a composition and/or formulation of the present disclosure to a subject. In certain embodiments, the method includes administering to a subject a composition or formulation that includes one or more types of iron oxide nanoparticles and stannous fluoride (SnF₂).

In certain embodiments, a composition and/or formulation of the present disclosure can be administered to the subject for a short time interval such as, but not limited, for a time period of less than about 10 minutes, less than about 9 minutes, less than about 8 minutes, less than about 7 minutes, less than about 6 minutes, less than about 5 minutes, less than about 4 minutes less, than about 3 minutes, less than about 2 minutes or less than about 1 minute.

An “effective amount,” as used herein, refers to an amount effective, at dosages and for periods of time necessary, to achieve the desired therapeutic or prophylactic result. For the prevention or treatment of disease, the appropriate amount, e.g., an effective amount, of a composition or formulation of the present disclosure will depend on the type of disease to be treated or prevented and the severity and course of the disease. Dosage regimens can be adjusted to provide the optimum therapeutic response.

In certain embodiments, the method can further include incubating the one or more iron oxide nanoparticles and SnF₂ for a predetermined time. In non-limiting embodiments, the predetermined time can be between 1 minute to 12 months. In non-limiting embodiments, the predetermined time for the incubation can be from about 1 minute to 5 hours, about 1 minute to 4 hours, about 1 minute to 3 hours, about 1 minute to 2 hours, about 1 minute to 60 minutes, about 1 minute to 50 minutes, about 1 minute to 40 minutes, about 1 minute to 30 minutes, about 1 minute to 20 minutes, or about 1 minute to 10 minutes. In non-limiting embodiments, the incubation time can be about 60 minutes. In non-limiting embodiments, the predetermined incubation time can be from a minute to a year. For example, the predetermined incubation time can be from about an hour to 12 months, from about an hour to 11 months, from about an hour to 10 months, from about an hour to 9 months, from about an hour to 8 months, from about an hour to 7 months, from about an hour to 6 months, from about an hour to 5 months, from about an hour to 4 months, from about an hour to 3 months, from about an hour to 2 months, from about an hour to 1 month, from about an hour to 30 days, from about an hour to 20 days, from about an hour to 10 days, from about an hour to 7 days, from about an hour to 6 days, from about an hour to 5 days, from about an hour to 4 days, from about an hour to 3 days, from about an hour to 2 days, from about an hour to 24 hours, from about an hour to 12 hours, from about an hour to 6 hours, from about an hour to 5 hours, from about an hour to 4 hours, from about an hour to 3 hours, or from about an hour to 2 hours. In certain embodiments, after the incubation, Sn²⁺ of the SnF₂ can be bound by carboxylate groups in a carboxymethyl-dextran coating of the one or more Fer nanoparticles.

In certain embodiments, the method can further include the administration of hydrogen peroxide, e.g., by the administration of a solution that includes hydrogen peroxide, to the subject. Alternatively or additionally, hydrogen peroxide can be present in the composition and/or formulation that includes the nanoparticles. For example, and not by way of limitation, hydrogen peroxide can be formulated in a gel-like product, e.g., toothpaste, using sodium percarbonate, where the gel-like product further includes one or more types of iron oxide nanoparticles. In certain embodiments, sodium percarbonate can be present within the composition and/or formulation to release hydrogen peroxide in the presence of water or when placed in the mouth. Such compositions and/or formulations can allow the release of hydrogen peroxide from the composition and/or formulation when contacted with an aqueous solution or when placed in the mouth, thereby allowing the reaction between the hydrogen peroxide and the types of iron oxide nanoparticles to occur in situ.

In certain embodiments, the method can further include performing a polymeric coating on the disclosed nanoparticles. In non-limiting embodiments, the Fer nanoparticles can include a polymeric coating, for example, and not by way of limitation, the polymeric coating can include chitosan, citrate, poly(acrylic acid) or dextran. In certain embodiments, the polymeric coating can be dextran or a modified dextran. For example, and not by way of limitation, the dextran can be cross-linked, aminated, carboxylated or modified with diethyl aminoethyl moieties. In certain embodiments, the coating of nanoparticles of the present disclosure can have a molecular weight from about 1 kDa to about 100 kDa, e.g., from about 1 kDa to about 90 kDa, from about 1 kDa to about 80 kDa, from about 1 kDa to about 70 kDa, from about 1 kDa to about 60 kDa, from about 1 kDa to about 50 kDa, from about 1 kDa to about 40 kDa, from about 1 kDa to about 30 kDa, from about 1 kDa to about 20 kDa, from about 1 kDa to about 10 kDa, from about 1 kDa to about 5 kDa, from about 5 kDa to about 100 kDa, from about 10 kDa to about 100 kDa, from about 20 kDa to about 100 kDa, from about 30 kDa to about 100 kDa, from about 40 kDa to about 100 kDa, from about 50 kDa to about 100 kDa, from about 60 kDa to about 100 kDa, from about 70 kDa to about 100 kDa, from about 80 kDa to about 100 kDa or from about 90 kDa to about 100 kDa.

In certain embodiments, Sn²⁺ of the SnF₂ can be bound by carboxylate groups in a carboxymethyl-dextran coating of one or more types of iron oxide nanoparticles. For example, iron oxide nanoparticles can stabilize SnF₂ through Sn²⁺ interactions with the carboxylate group in the carboxymethyl-dextran coating of the nanoparticles. The inclusion of SnF₂ can enhance the catalytic (peroxidase-like) activity of Fer under pathological conditions (acidic pH) but not at physiological pH (pH>6.5), thereby increasing its specificity and antibiofilm activity in cariogenic conditions. Conversely, the inclusion of carboxymethyl-dextran coating increases stability of SnF₂ in aqueous solution while also serving as Sn and fluoride delivery system.

In certain embodiments, the method can further include the administration of an effective amount of additional fluoride. In certain embodiments, fluoride can be present in the composition and/or formulation that includes the nanoparticles with SnF₂ and/or hydrogen peroxide. For example, and not by way of limitation, fluoride can be formulated in a gel-like product, as disclosed above, where the gel-like product further includes one or more nanoparticles with SnF₂ and/or hydrogen peroxide. In certain embodiments, the additional fluoride can be present within a composition and/or formulation of the present disclosure at a concentration of about 10 parts per million (ppm) to about 10,000 ppm, e.g., about 5,000 ppm.

In certain embodiments, a composition or formulation of the present disclosure can be administered to the subject one time or over a series of treatments. In certain embodiments, several divided doses can be administered daily, or the dose can be proportionally reduced as indicated by the exigencies of the therapeutic situation. For example, but not by way of limitation, the compositions and formulations disclosed herein can be administered to a subject twice every day, once every day, once every two days, once every three days, once every four days, once every five days, once every six days, once a week, once every two weeks, once every three weeks, once every month, once every two months, once every three months, once every six months or once every year. In certain embodiments, a composition or formulation of the present disclosure, e.g., a composition that includes one or more Fer nanoparticles with SnF₂, can be administered to a subject twice every day. In certain embodiments, a composition that includes one or more Fer nanoparticles, e.g., in a mouth rinse formulation, can be administered to a subject once or twice every day, followed by the administration of H₂O₂ once or twice every day, once every two days, once every three days, once every four days, once every five days, once every six days or once a week. In certain embodiments, a composition that includes one or more Fer nanoparticles with SnF₂ and sodium percarbonate, which in turn, generates H₂O₂, can be administered to a subject, e.g., in a gel-based formulation, once or twice every day, once every two days, once every three days, once every four days, once every five days, once every six days or once a week.

In certain embodiments, the disclosed composition can include citric acid. In non-limiting embodiments, the disclosed composition can include the citric acid ranging from about 1 μg/ml to about 10000 μg/ml, from about 1 μg/ml to about 5000 μg/ml, from about 1 μg/ml to about 1000 μg/ml, from about 1 μg/ml to about 900 μg/ml, from about 1 μg/ml to about 800 μg/ml, from about 1 μg/ml to about 700 μg/ml, from about 1 μg/ml to about 600 μg/ml, from about 1 μg/ml to about 500 μg/ml, from about 1 μg/ml to about 400 μg/ml, from about 1 μg/ml to about 300 μg/ml, from about 1 μg/ml to about 10000 μg/ml, from about 1 μg/ml to about 10000 μg/ml, from about 1 μg/ml to about 250 μg/ml, from about 1 μg/ml to about 200 μg/ml, or from about 1 μg/ml to about 125 μg/ml. In non-limiting embodiments, the disclosed composition can include citric acid ranging from about 125 μg/ml to about 1000 μg/ml.

In certain embodiments, the disclosed iron oxide nanoparticles (IONP) can be coated with citric acid. In non-limiting embodiments, the IONPs can be premixed and/or incubated with citric acid.

In certain embodiments, the addition of citric acid can improve stability and catalytic activities of IONPs as well as aqueous solubility of IONPs/Fer and SnF₂. For example, the size of Fer/SnF₂ does not change appreciably when mixed with citric acid, thereby demonstrating stability. However, under certain ratios of Fer to SnF₂, size can increase remarkably in the absence of citric acid. In non-limiting embodiments, the catalytic activity of Fer can increase in a dose-dependent manner when citric acid is added to the composition.

The present disclosure further provides methods for the prevention of bacterial growth in a biofilm. In certain embodiments, such methods can include contacting a surface having a biofilm with an effective amount of a composition and/or formulation, disclosed herein, that includes one or more types of iron oxide nanoparticles. In certain embodiments, the one or more types of nanoparticles bind to the surface and catalyze H₂O₂ and/or delivery SnF₂ to inhibit bacterial growth within the biofilm.

The present disclosure further provides methods for preventing the formation of a biofilm on a surface. In certain embodiments, a method for preventing the formation of a biofilm on a surface can include treating a surface that is “at risk” for biofilm development with an effective amount of a composition and/or formulation, disclosed herein, that includes one or more iron nanoparticles. In certain embodiments, the method can further include contacting the “at risk” surface with H₂O₂. For example, and not by way of limitation, an effective amount of a composition and/or formulation, disclosed herein, that includes one or more iron oxide nanoparticles can be coated on the surface, e.g., by spraying or painting, or incorporated into materials, e.g. denture materials or restorative materials/resins. Surfaces that are “at risk” for developing a biofilm include, but are not limited to, apatitic surfaces, e.g., bone and tooth surfaces, endodontic canals, implant surfaces, medical device surfaces, e.g., catheters and instruments, and industrial and naval surfaces, e.g., pipe and ship hull surfaces. In certain embodiments, the surface can be the interior and/or exterior surface of a medical device and industrial and/or naval material.

The presently disclosed subject matter further provides methods for preventing tooth demineralization. In certain embodiments, a method for the prevention of demineralization can include contacting a tooth-enamel or an apatitic (e.g., bone) surface having a biofilm with an effective amount of a composition that includes one or more iron nanoparticles. In certain embodiments, the one or more types of iron oxide nanoparticles bind to the surface to inhibit and/or prevent enamel or apatitic dissolution. In non-limiting embodiments, the disclosed nanoparticles can deliver SnF₂ to the target biofilm and/or the enamel surface. The treatment of the disclosed compositions can create a protective outer layer in the enamel enriched with Sn, iron, and fluoride that can protect against enamel demineralization while also serving as a reservoir for Sn, which can serve as an antibacterial layer at the target surface (e.g., tooth surface).

By creating a protective layer and depositing on the mineralized tissues, it can reduce dental sensitivity caused by worn enamel and exposed dentin.

In certain embodiments, the disclosed subject matter can be administered or delivered through an applicator. An example applicator can be a dual-compartment applicator. The dual-compartment applicator can include a first chamber containing Fer/SnF₂ and a second chamber containing H₂O₂. In non-limiting embodiments, the solutions in each chamber can be mixed as a user presses the chambers into a single nozzle. The solutions can be mixed in real-time prior to application.

The presently disclosed subject matter further provides methods for the treatment and elimination of biofilms and/or the prevention of biofilm formation on a surface of a medical device or an industrial and/or naval material. In certain embodiments, the method can include contacting a medical device, e.g., catheters, implants, artificial joints, tubing, any implanted devices, or an industrial and/or naval material, e.g., a pipe, containers, reactors, turbines or ship hulls with a composition or formulation disclosed herein. In certain embodiments, the method can include contacting a surface of a medical device or industrial material with a composition or formulation that includes the nanoparticles with SnF₂. In certain embodiments, the method can further include contacting the surface of a medical device or industrial material with H₂O₂. In certain embodiments, a composition or formulation of the present disclosure can be incorporated into a material for manufacturing a medical device or an industrial and/or naval material to prevent, minimize and/or reduce the formation of a biofilm on a surface of the medical device or industrial and/or naval material.

### EXAMPLES

### Example 1: Ferumoxytol Nanoparticles Stabilize Stannous Fluoride for Synergistic Biofilm Disruption and Tooth-Decay Prevention

Antibiofilm activity of ferumoxytol (Fer) in combination with SnF₂ in vitro: Fluoride is widely used as a gold standard anticaries agent, but it does not provide full protection, especially in controlling the formation of dental biofilms that causes demineralization of tooth enamel. Despite its limited antibiofilm activity, sodium fluoride (NaF) can affect bacterial glycolysis and acid tolerance, whereas stannous fluoride (SnF₂) provides stronger antibacterial activity imparted by Sn²⁺ ions. First, the antibiofilm activity of both NaF and SnF₂ (1000 ppm of F, the typical concentration in oral care formulations) was tested. SnF₂ can significantly inhibit the growth of S. mutans (cariogenic pathogen) and reduce biomass when compared with NaF, indicating that SnF₂ is a much more effective biofilm treatment. Afterward, NaF or SnF₂ were combined with Fer (1 mg of Fe/ml, an effective antibiofilm concentration) in the presence of 1% H₂O₂. The data show that the combination of Fer with SnF₂ has significantly greater antibacterial activity and reduces the biofilm biomass (dry-weight) more effectively than either alone or the combination of Fer and NaF. Since the combination of NaF with Fer is ineffective, SnF₂ can interact with Fer for enhanced effects.

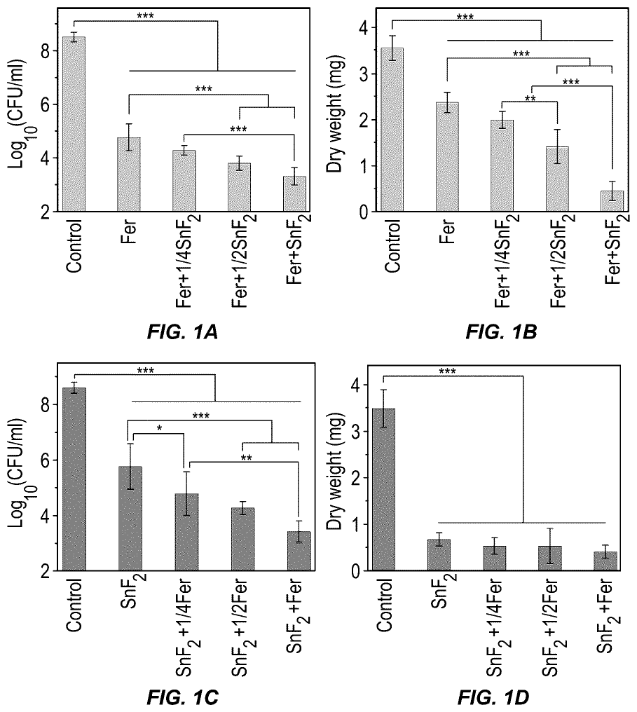

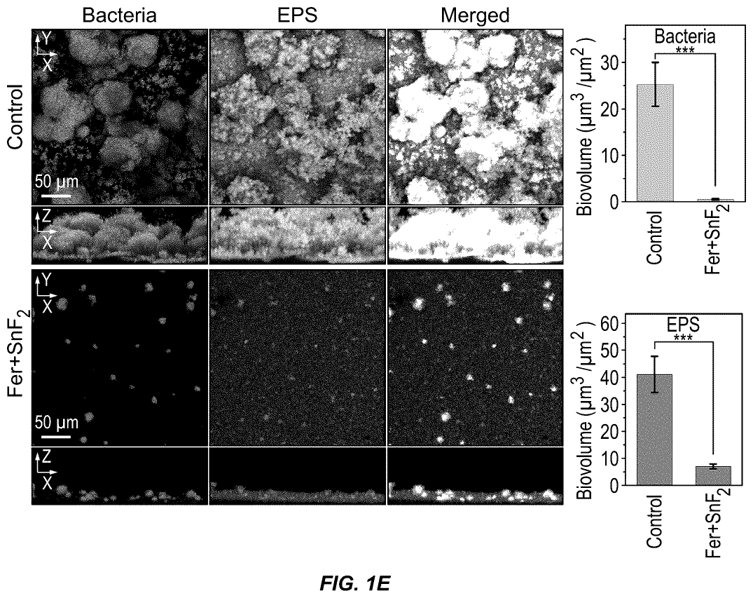

Antibiofilm activity was evaluated using different concentrations of Fer and SnF₂ in the presence of H₂O₂ using the saliva-coated hydroxyapatite disc (tooth enamel mimic) model in the presence of sucrose. Initially, Fer (1 mg of Fe/ml) was mixed with various concentrations of SnF₂ (0-250 ppm of F), and the number of viable cells and biomass were determined. As expected, Fer displayed a strong biocidal effect against S. mutans biofilm (>3-log reduction of viable cells; FIG. 1A) while also reducing biomass (FIG. 1B). When Fer was mixed with increasing concentrations of SnF₂, both the antibacterial activity and the inhibitory effect on the biomass enhanced in a dose-dependent manner, indicating that SnF₂ can help improve the antibiofilm efficacy of Fer.

In addition, various concentrations of Fer (0-1 mg of Fe/ml) were also mixed with SnF₂ (250 ppm of F). When combined with Fer, the antibacterial effect against S. mutans significantly increased in a dose-dependent manner, resulting in >5-log reduction of viable cells compared to control when the concentration of Fer reached 1 mg of Fe/ml. The combination of Fer and SnF₂ was ˜2500-fold more effective in killing S. mutans cells than SnF₂ alone (FIG. 1C), suggesting a synergistic effect to potentiate the killing efficacy of the agents. Notably, SnF₂ (250 ppm of F) alone can substantially reduce biomass (FIG. 1D), and in combination with increasing amounts of Fer, did not enhance the bioactivity, suggesting that SnF₂ disrupts the accumulation of extracellular polysaccharides (EPS) that provide the bulk of biofilm dry weight, congruent with SnF₂'s inhibitory effects against EPS-producing glucosyltransferases secreted by S. mutans. The antibiofilm activity of the combination of Fer (1 mg of Fe/ml) and SnF₂ (250 ppm of F) was further confirmed with high-resolution confocal microscopy (FIG. 1E), whereby the agents impaired the accumulation of bacterial cells and the development of EPS α-glucan matrix.

### Characterization of the Combination of Fer and SnF₂

Given the enhanced efficacy of the combination of Fer and SnF₂, its physicochemical properties were assessed. The hydrodynamic diameters of Fer and Fer mixed with SnF₂ were (23.8±0.9) nm and (23.9±1.1) nm, whereas the zeta potentials were (−8.4±1.4) mV and (−10.1±1.4) mV, respectively (Table 1). Overall, mixing Fer with SnF₂ did not seem to affect the size or zeta potential.

TABLE 1
The average hydrodynamic diameter and zeta
potential of Fer and Fer + SnF₂.

| Samples | Fer | Fer + SnF₂ |
|---|---|---|
| Hydrodynamic Diameter (nm) | 23.8 ± 0.9 | 23.9 ± 1.1 |
| Zeta Potential (mV) | −8.4 ± 1.4 | −10.1 ± 1.4 |

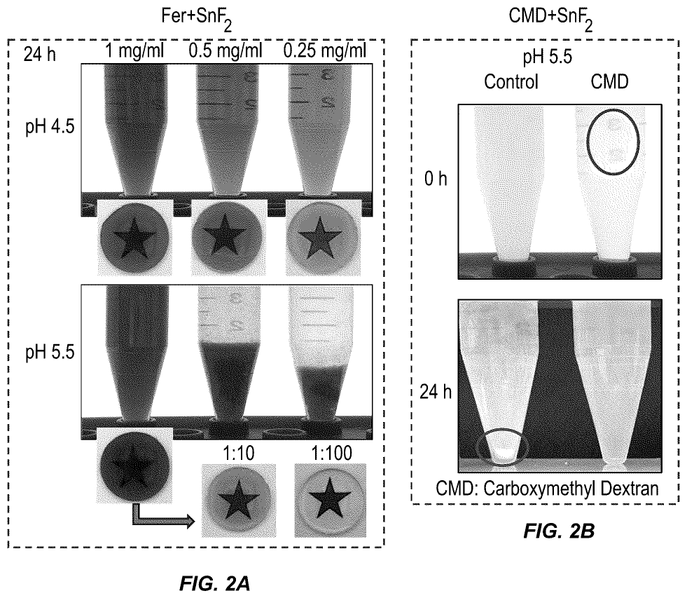

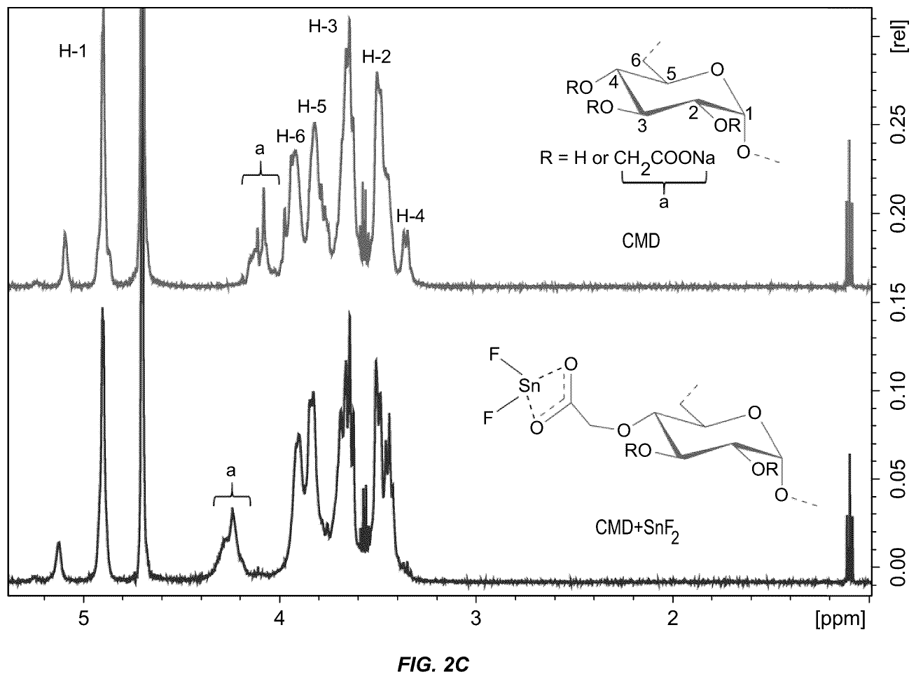

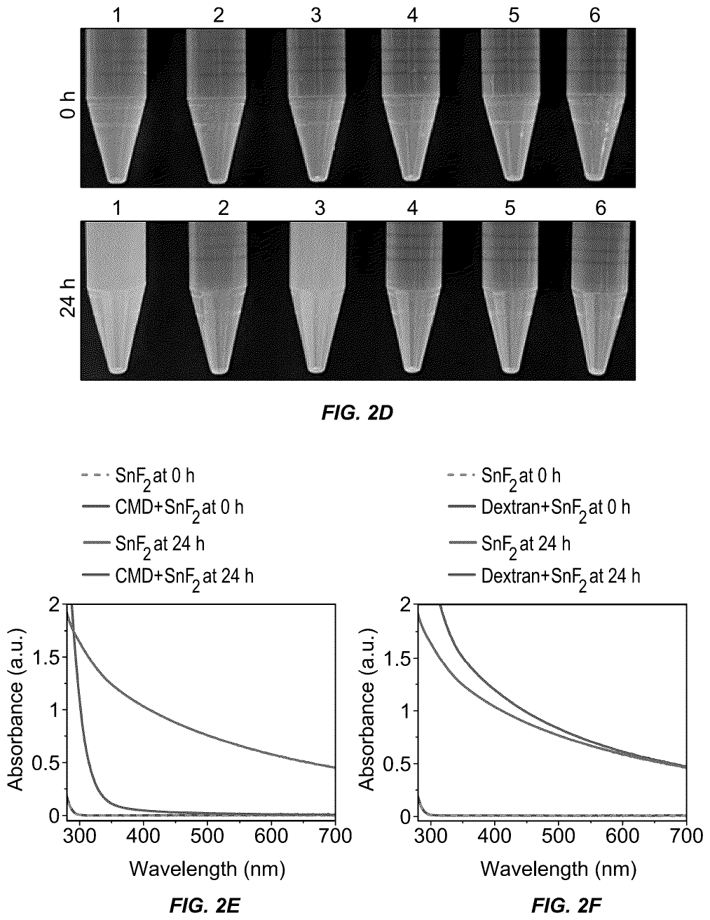

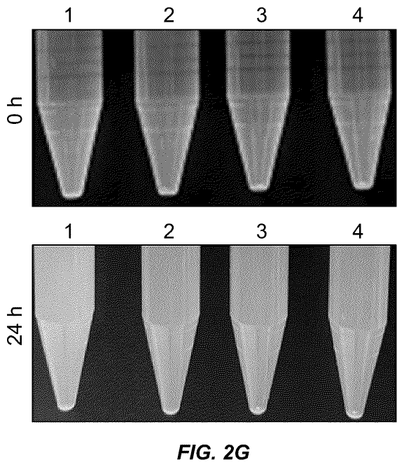

It is noteworthy that SnF₂ has limited stability in aqueous solutions owing to its high susceptibility to hydrolysis and oxidation, requiring chemical additives (e.g., chelating agents) or water removal, which can reduce fluoride bioavailability. SnF₂ was stable in aqueous solutions containing Fer. To further investigate the stability of SnF₂ in the presence of Fer, SnF₂ (250 ppm of F) was mixed with increasing amounts of Fer in 0.1 M sodium acetate buffer at different pH values. The solution containing SnF₂ mixed with Fer was limpid after 24 h in sodium acetate buffer at pH 4.5 (FIG. 2A). However, aggregation occurred when SnF₂ was mixed with lower amounts of Fer (0.5 mg of Fe/ml or 0.25 mg of Fe/ml) at pH 5.5 (FIG. 2A), indicating that SnF₂ is more stable when mixed under acidic conditions (pH 4.5), and its stability is dependent on the concentration of Fer. Recent data show that the combination is also stable in water.

The Fer core is coated with carboxymethyl-dextran (CMD). Thus, whether SnF₂ can interact with CMD was assessed. SnF₂ alone or mixed with CMD was incubated in 0.1 M sodium acetate buffer pH 5.5 for 24 h. Immediate aggregation of SnF₂ dissolved in sodium acetate buffer (FIG. 2B) was observed, whereas the solution was limpid when mixed with CMD both before and after 24 h incubation. In addition, the mixture of SnF₂ and CMD was characterized by ¹H NMR (FIG. 2C). In the CMD spectrum, the anomeric proton (H1) in the C1 position was identified at 4.9 ppm, and protons (H2-H6) at the C2-C6 positions were detected at 3.2-4.0 ppm. The peak at 4.0-4.2 ppm (denoted as “a”) is attributed to the protons of the carboxymethyl moieties, as determined previously. After CMD was mixed with SnF₂, a clear shift in peak “a” was observed when compared to that of CMD alone. This suggests that Sn²⁺ binds to the carboxymethyl moieties of CMD, which can account for the enhanced stability of SnF₂ with Fer. Note that similar ¹H NMR studies of SnF₂ and Fer are not possible due to the superparamagnetic nature of Fer interfering with ¹H NMR measurements.

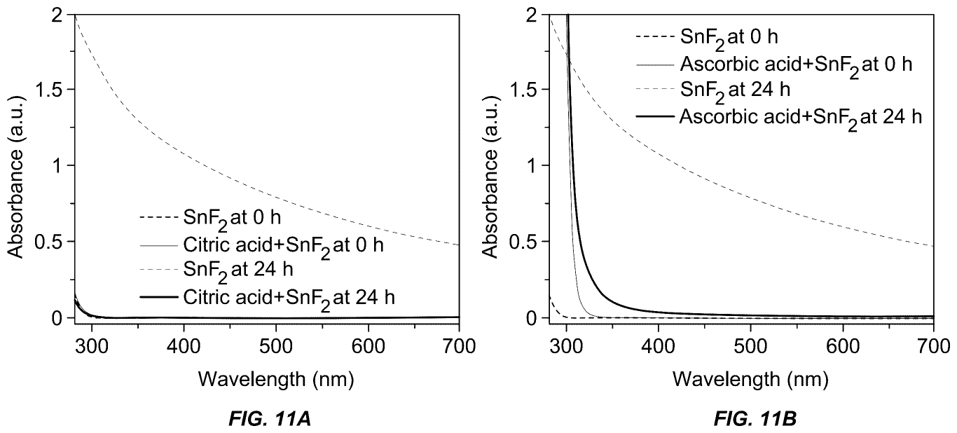

In order to further investigate the effects of CMD on SnF₂ stability, it was compared with several control materials, i.e., dextran (a similar polymer to CMD, but without carboxylic acid groups), as well as citric acid, L-ascorbic acid, and polyacrylic acid (PAA), all entities that all contain carboxylic acid groups). Dextran did not enhance the stability of SnF₂, whereas each material that contains carboxylic acid groups did enhance stability (FIGS. 2D-2F and FIG. 11). The Fer formulation also contains mannitol, which is an antioxidant. Since antioxidants can prevent the oxidation of SnF₂, SnF₂ was added to various amounts of mannitol (1-10 mg/ml). Surprisingly, any noticeable change in the stability of SnF₂ was detected even with excess amounts of mannitol (10 mg/ml) (FIG. 2G), implying that mannitol does not have any noticeable effect on enhancing the stability of SnF₂

### Catalytic Activity of Fer in Combination with SnF₂

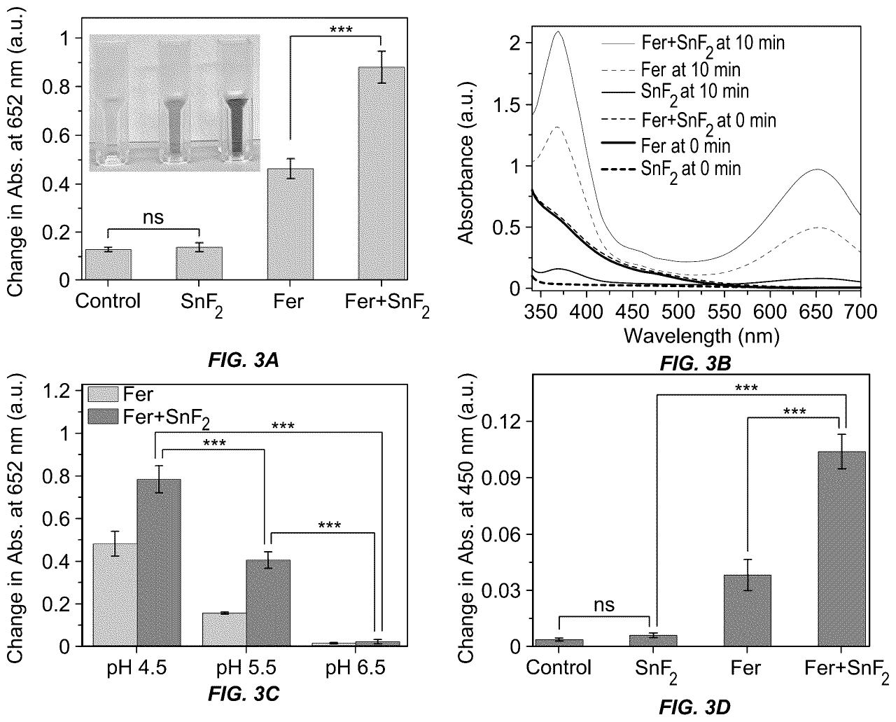

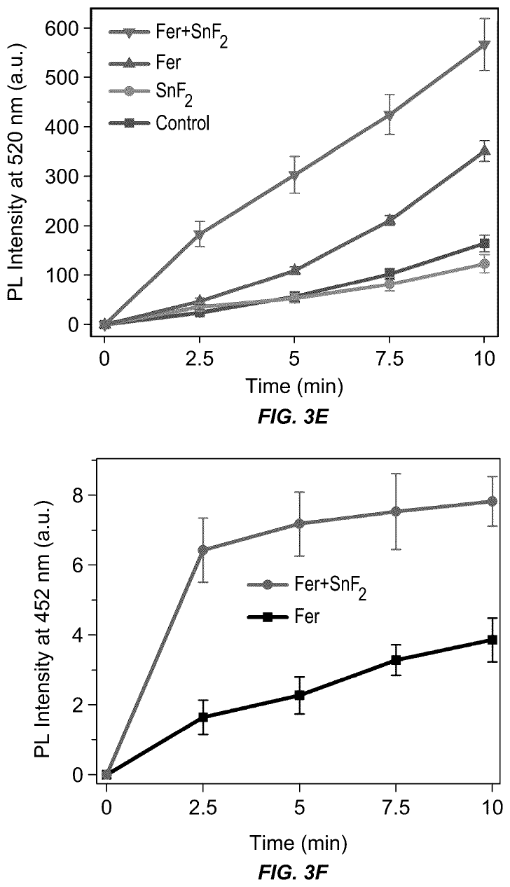

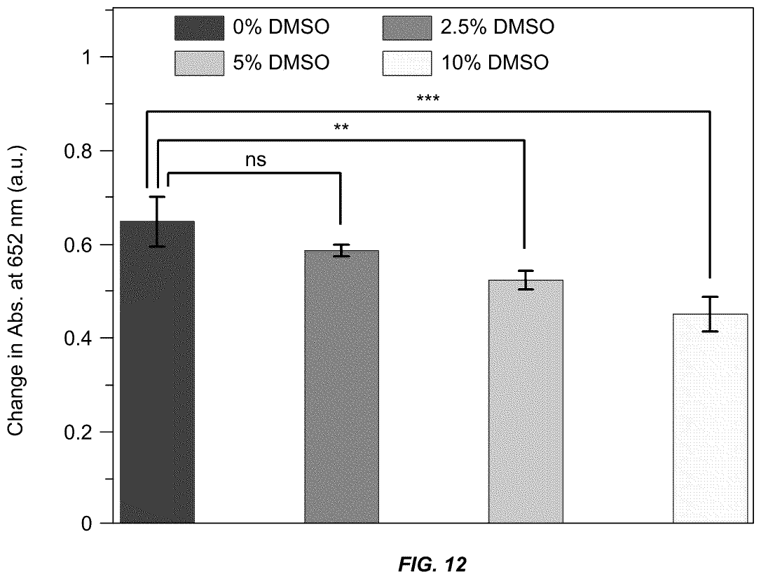

To explore whether SnF₂ could produce ROS, the 3,3′,5,5′-tetramethylbenzidine (TMB) colorimetric assay was used for peroxidase-like activity following a previously published protocol, with some modifications. TMB is a chromogenic compound that yields a blue color upon oxidation with an absorption peak at 652 nm in the presence of H₂O₂. As shown in FIGS. 3a and b, SnF₂ alone did not produce a noticeable amount of ROS. In contrast, the catalytic activity of Fer increased significantly after combining with SnF₂, as demonstrated by increased colorimetric reaction (FIGS. 3A and 3B), suggesting that SnF₂ enhanced the catalytic activity of Fer. Adding various amounts of DMSO (a well-known quencher of ·OH) to the mixture of Fer+SnF₂ resulted in reduced absorption of TMB at 652 nm (FIG. 12), confirming that part of the ROS produced is ·OH.

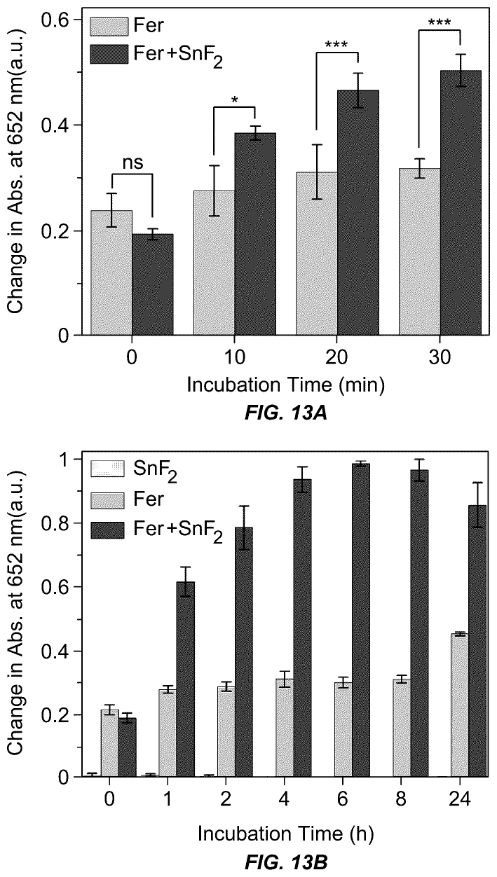

Notably, the enhancement of ROS production in the presence of SnF₂ is pH and incubation time-dependent. The highest catalytic activity was observed at pH 4.5 (FIG. 3C). The greater ROS production at acidic pH conditions (characteristic of pathological conditions associated with dental caries) and the minimal ROS generation close to neutral (physiological) pH suggests that the Fer exhibits peroxidase-like activity that is enhanced when mixed with SnF₂. Moreover, significant amounts of ROS can be detected within 10 min incubation (FIG. 13A), gradually increasing to reach the highest level at 6 h (FIG. 13B), indicating the importance of the incubation time while mixing Fer and SnF₂.

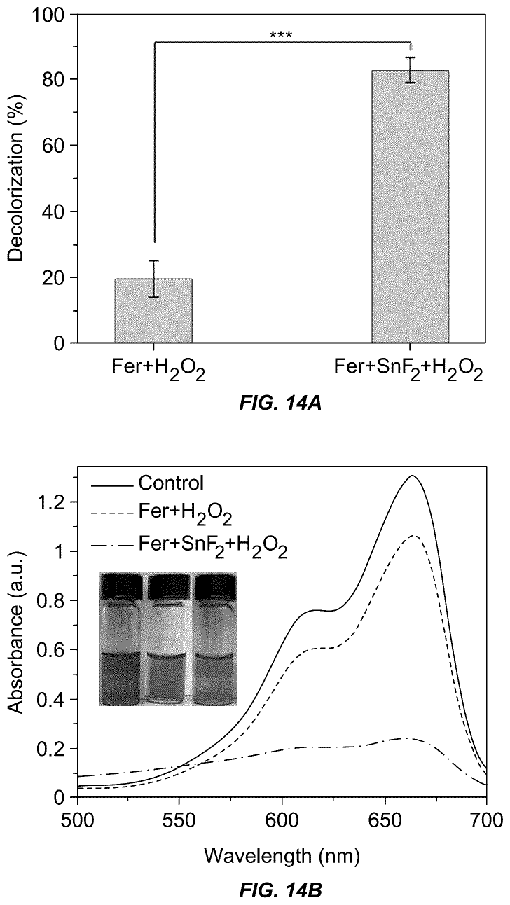

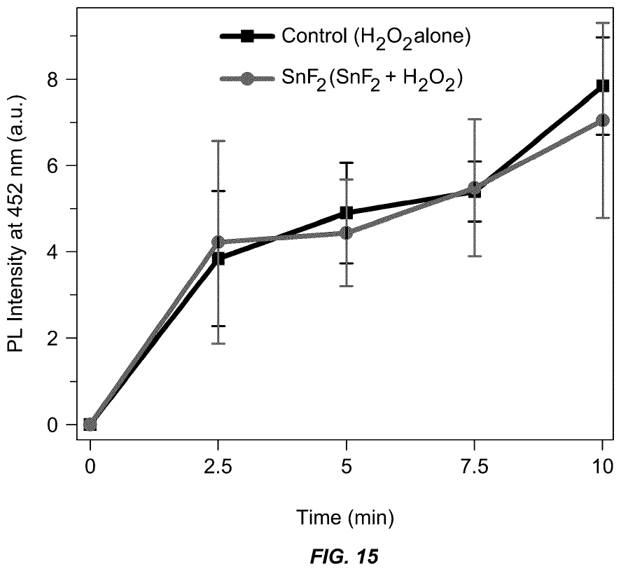

To further confirm the enhancement of the peroxidase-like activity of the Fer in the presence of SnF₂, a multi-pronged approach was used. First, OPD, a colorless substrate, which yields an oxidized product with a characteristic yellow color when reacting with ROS with an absorption peak at 450 nm, was used. As expected, the catalytic activity of Fer increased markedly after adding SnF₂ as compared to Fer alone and SnF₂ alone (FIG. 3D). Second, the decolorization of methylene blue to complement the colorimetric assay was evaluated. Remarkably, Fer+SnF₂ was 4-fold more effective in decoloring methylene blue than Fer (FIG. 14). Third, ROS production was evaluated by the photoluminescence (PL) method using DCFH-DA as a ROS tracking probe. DCFH-DA (a nonfluorescent molecule) yields a fluorescent molecule DCF in the presence of ROS. As depicted in FIG. 3E, the PL intensity at 520 nm increased to a greater extent after combining Fer with SnF₂. The generation of ·OH was compared by PL method using coumarin as a probe molecule, which produces a highly fluorescent 7-hydroxycoumarin in the presence of ·OH. As seen in FIG. 3F, the PL intensity at 452 nm increased to a greater extent after combining SnF₂ as compared to Fer alone, justifying the higher amount of ·OH production and further confirming that SnF₂ enhanced the catalytic activity of Fer. However, SnF₂ alone did not produce a noticeable amount of ·OH (FIG. 15), consistent with all the previous observations.

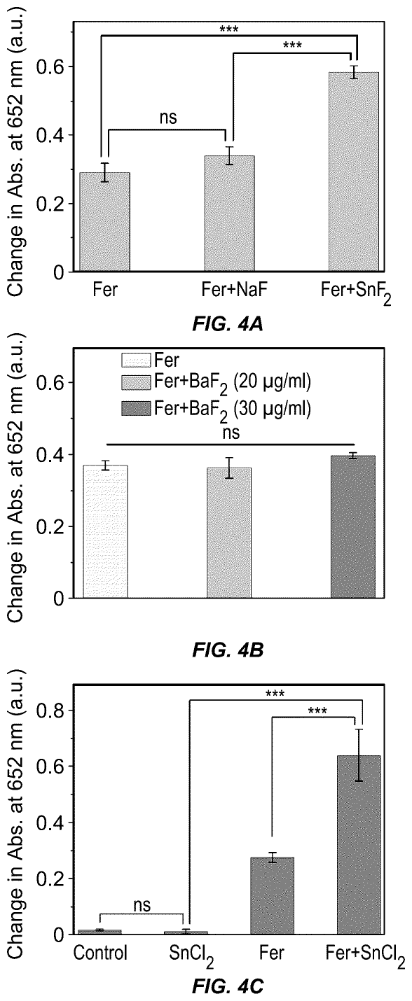

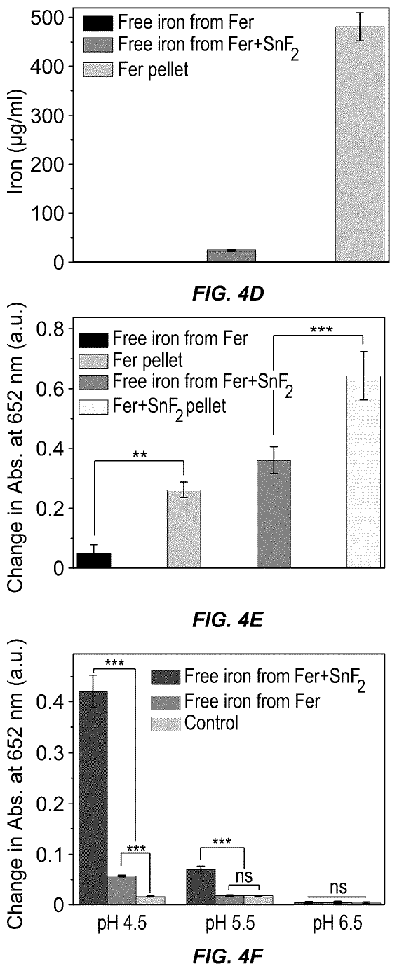

Next, whether the augmented catalytic activity arises from different fluoride or stannous salts was assessed. SnF₂ was replaced with NaF, a commonly used fluoride salt in oral care formulations, or barium fluoride (BaF₂). Neither NaF nor BaF₂ increased the catalytic activity of Fer (FIGS. 4A and 4B), suggesting that F⁻ does not play a crucial role in enhancing the ROS production performance of Fer. Conversely, SnCl₂ was used to evaluate whether Sn ions play a role in strengthening the ROS generation capability of Fer. SnCl₂ enhanced the catalytic activity of Fer (FIG. 4C), indicating that Sn ions can play a dominant role in increasing the catalytic performance of Fer. Taken together, these findings support that SnF₂ can boost the catalytic ability of Fer, indicating that Fer and SnF₂ combination is an effective ROS-generating therapy that can target biofilms under pathological (acidic) conditions.

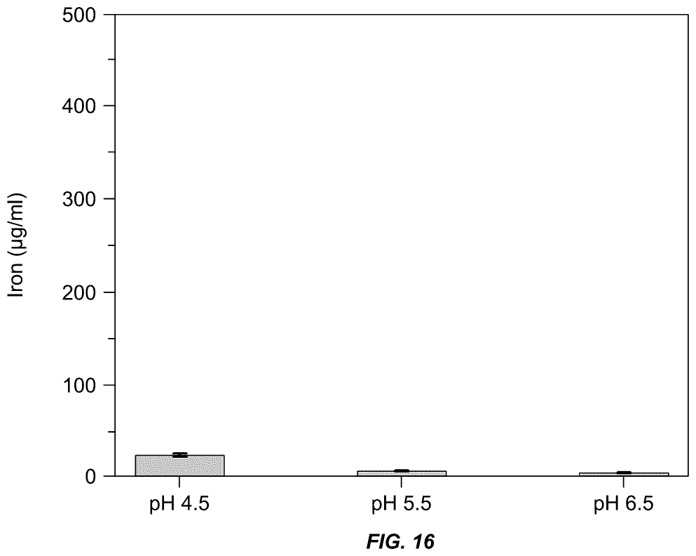

Whether Fer released iron ions when combined with SnF₂ at acidic pH (4.5) was assessed using ICP-OES. As depicted in FIG. 4D, the presence of SnF₂ slightly increased iron ions released from Fer. Interestingly, even though Fer releases a low percentage of iron ions when mixed with SnF₂, their catalytic activity is significant at pH 4.5 (FIG. 4E), suggesting that leached iron contributes, to some extent, to the enhanced ROS generation via the homogeneous Fenton reaction. The catalytic activity of the pelleted Fer+SnF₂ (devoid of free iron ions) was also assessed. Surprisingly, the catalytic activity of the Fer+SnF₂ pellet is substantially higher than that of the Fer pellet (FIG. 4E). The reason why the pellet of Fer+SnF₂ generates much higher levels of ROS as compared to Fer alone remains unclear, although it is possible that Sn ions could accelerate the Fe²⁺/Fe³⁺ redox cycles. It is noteworthy that the amount of leached irons from Fer+SnF₂ formulation at circumneutral pH is very low (FIG. 16), and there is little to no catalytic activity of the leached iron at pH 6.5 (FIG. 4F), which reduces the unwanted toxicity to normal cells and commensal bacteria at the physiological pH in the oral cavity (˜pH 6.8). Conversely, the iron leached from Fer+SnF₂ at acidic pH values could provide an added benefit. Iron ions have shown cariostatic effects as they can precipitate on the surface of enamel and promote the adsorption of phosphate and calcium ions, thereby reducing enamel demineralization.

Altogether, the increased stability of SnF₂ in aqueous solutions is mediated at least in part via interactions with CMD, which can be important for both fluoride bioavailability and fluoride delivery. Unexpectedly, the presence of SnF₂ boosts the ROS generation capability of Fer at acidic pH that can not only enhance the antibiofilm potency but also reduce demineralization. This synergistic Fer and SnF₂ combination provides a potent yet pH-dependent ROS-based therapy with enhanced antimicrobial fluoride stability that could prevent the onset of dental caries in vivo.

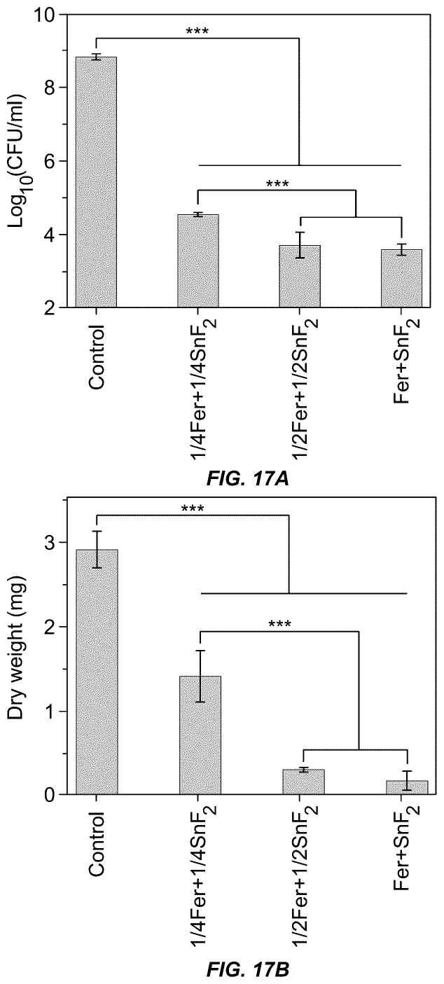

Impact of Fer/SnF₂ on caries development and host-microbiota/tissues in vivo: Topical applications of Fer and SnF₂ in vivo were further assessed using a rodent model that mimics the characteristics of severe human caries. Rat pups were infected with S. mutans (oral bacterial pathogen) and fed a sugar-rich diet. In this model, tooth enamel progressively develops caries lesions (analogous to those observed in humans), proceeding from initial areas of demineralization to severe lesions characterized by enamel structure damage and cavitation. The test agents were topically applied twice daily with 1 min exposure time to mimic the clinical use of a mouthwash. After 5 weeks of treatment, the incidence and severity of caries lesions were evaluated. A reduced concentration of the combination of Fer (0.25 mg of Fe/ml) and SnF₂ (62.5 ppm of F) was included since the lower amounts were capable of significantly killing the bacteria (p<0.01) and reducing biomass (p<0.05) compared to control group (FIG. 17).

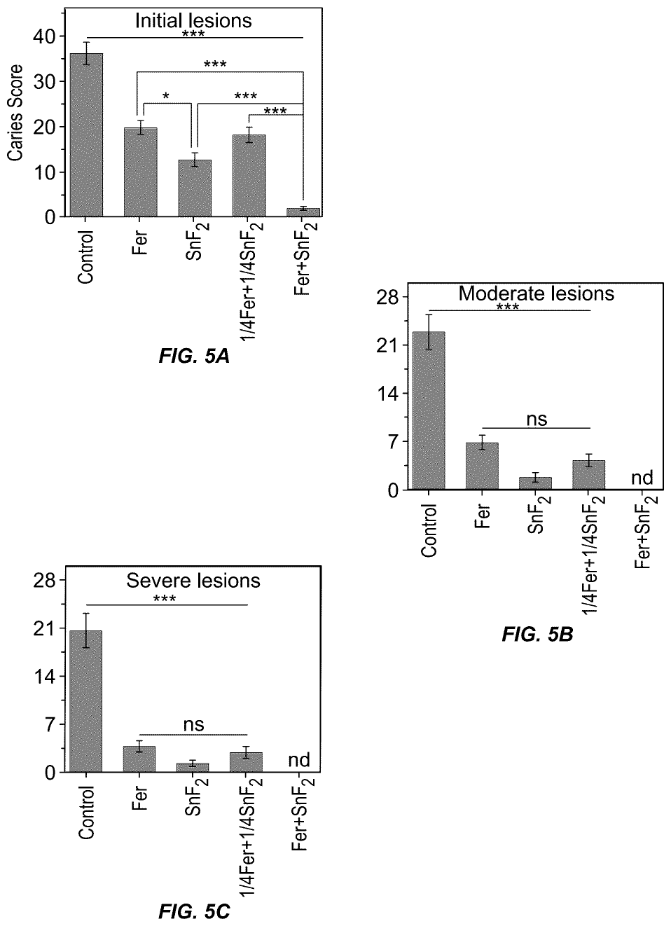

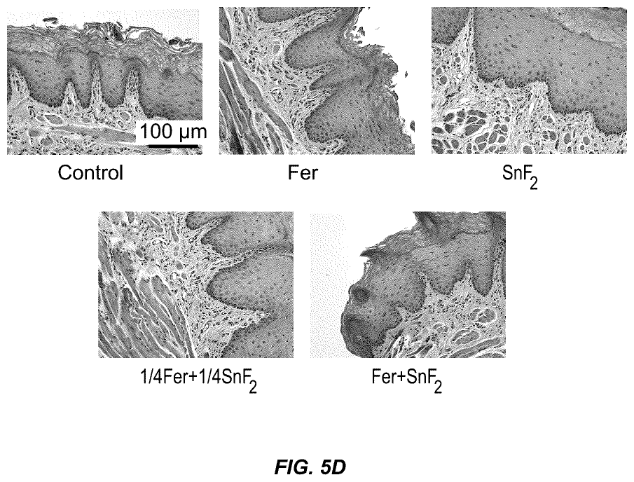

Quantitative caries scoring analyses revealed that the treatment of Fer in combination with SnF₂ was exceptionally effective in preventing caries development with higher efficacy than either alone (p<0.001) (FIG. 5A). It nearly abrogated caries initiation and completely blocked extensive caries lesions, thus preventing the onset of cavitation altogether (FIGS. 5B and 5C). The efficacy of the lower dosage of Fer and SnF₂ treatment was significantly greater than the control group (p<0.001) and not significantly different from Fer (1 mg of Fe/ml) or SnF₂ (250 ppm of F) treatment alone. This demonstrates that the combination of Fer and SnF₂ has a synergistic effect for efficient biofilm treatment and caries prevention in vivo. Histopathological analysis of gingival tissues revealed no cytotoxicity observed, such as proliferative changes, vascularization issues, necrosis, or acute inflammatory responses, suggesting biocompatibility of Fer and SnF₂ treatment (FIG. 5D).

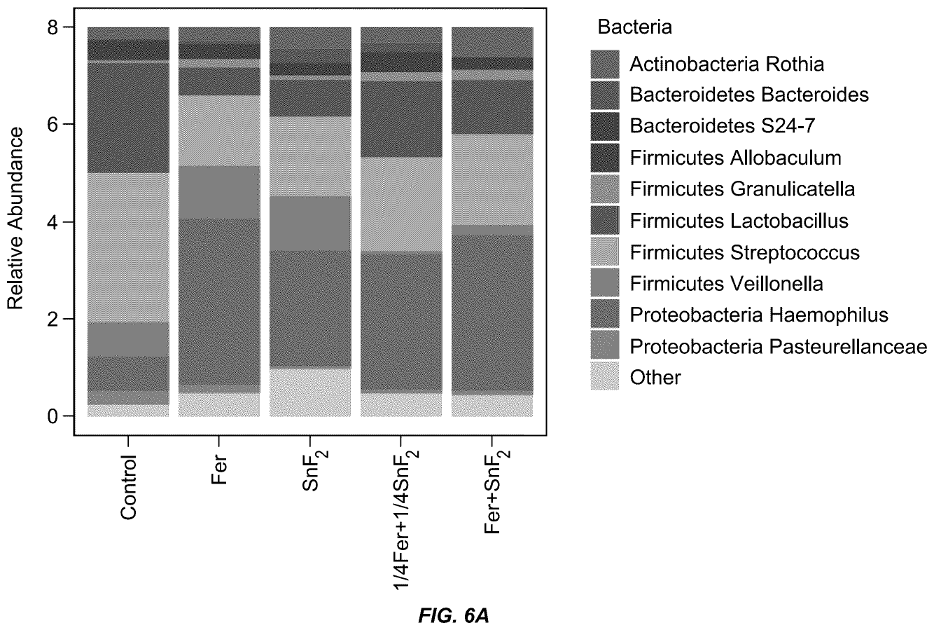

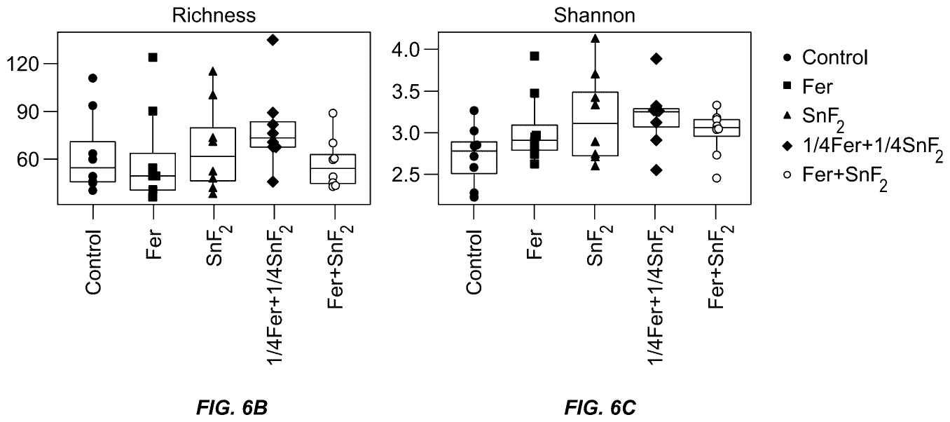

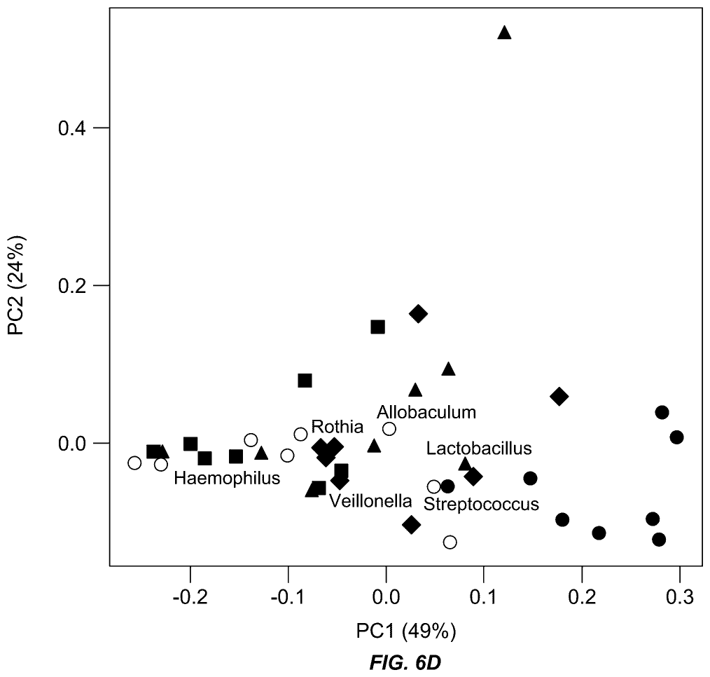

The effects of Fer and SnF₂ on oral microbiota were also evaluated, and all treatment groups showed similar microbiota composition (FIG. 6A) with no significant differences in alpha diversity among each group (FIGS. 6B and 6C, p>0.05, Willcox test). Furthermore, weighted Unifrac distances analyzed of principal coordinate analysis (PCoA) by treatment groups revealed that Fer and SnF₂ treatment group has a similar composition with the lowest dispersion (FIG. 6D), indicating no deleterious effects on the oral microbiota diversity (p>0.05, PERMANOVA). Collectively, the data show that the combination was substantially more potent than either alone, whereas a lower concentration of agents in combination was as effective as each alone at full strength, indicating a synergistic effect between Fer and SnF₂ in vivo without deleterious effects on the host tissues and oral microbiota diversity.

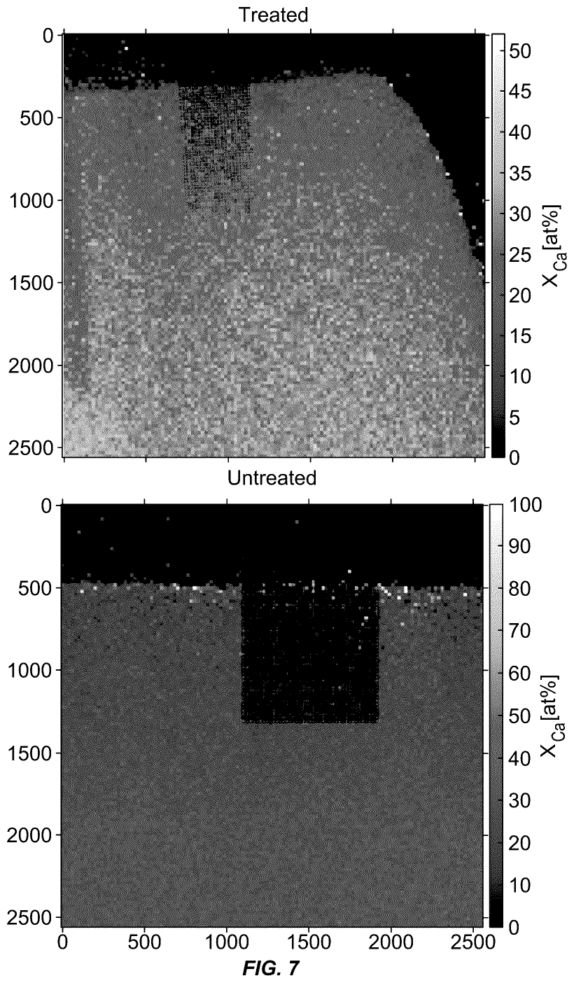

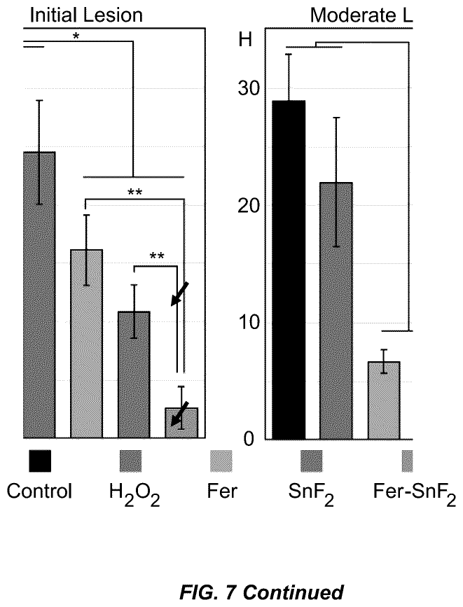

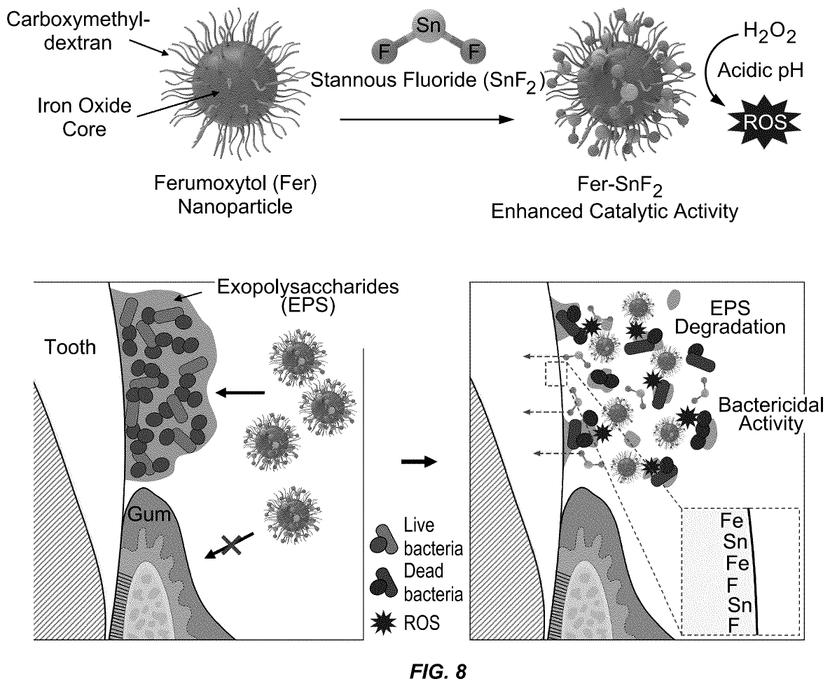

FIG. 7 provides enamel surface analysis showing the formation of a protective layer. The disclosed nanoparticles can deliver SnF₂ to the target biofilm and/or the enamel surface. FIG. 7 shows that the treatment of the disclosed compositions can create a protective outer layer in the enamel enriched with Sn, iron, and fluoride that can protect against enamel demineralization while also serving as a reservoir for Sn, which can serve as an antibacterial layer right at the tooth surface. FIG. 8 schematically depicts the above-described features of the Fer-SnF₂ combination. First, SnF₂ binds to Fer, which results in increased stability/solubility in aqueous solution and enhanced peroxidase like activity. Second, the combination results in drastically enhanced bacterial killing and EPS degradation. Third, the combination protects against demineralization and created a protective antibacterial/remineralizing outer layer, further averting adverse consequences of oral infections.

A remarkable synergy between ferumoxytol (Fer) nanoparticles and stannous fluoride (SnF₂) was observed in enhancing antibiofilm and anticaries efficacy (which did not occur with other fluoride sources, i.e., sodium fluoride (NaF) or with either SnF₂ or Fer alone). Fer can stabilize SnF₂ through Sn²⁺ interactions with the carboxylate group in the carboxymethyl-dextran coating of the nanoparticle. Conversely, the inclusion of SnF₂ significantly enhanced the catalytic (peroxidase-like) activity of Fer under pathological conditions (acidic pH) but not at physiological pH (pH>6.5), thereby increasing its specificity and antibiofilm activity in cariogenic conditions. The combination is far more effective than either treatment and completely halts caries lesions and cavitation in a severe rodent caries model, an outcome not seen before, without adverse effects on the surrounding host tissues and oral microbiota. Ultrastructure analysis revealed that fluoride, iron, and tin are detected in the outer layers of the enamel, suggesting co-delivery and chemical incorporation and creation of a protective outer layer onto the tooth surface. Notably, comparable therapeutic effects were achieved even at 4 times lower fluoride concentration, opening the possibility of a therapy that uses lower doses and operates through multiple mechanisms of action. This approach could lead to high therapeutic_efficacy for susceptible individuals prone to cariogenic biofilm accumulation while modulating enamel physicochemical properties.

Despite promising results, there are some limitations but also opportunities for further research. Although our preliminary study suggests that the COOH group is playing a key role in enhancing the stability of SnF₂, additional analyses are needed to further understand the physicochemical interactions between SnF₂ and Fer. Additional investigation is also required to elucidate the exact mechanisms by which ROS generation is enhanced by SnF₂. Interestingly, fluoride, iron, and tin can act in concert to enhance enamel resistance against demineralization, indicating a novel mechanism for caries protection that needs to be investigated in detail. Additional studies on the long-term stability of SnF₂, as well as full toxicity studies can be carried out to determine the long-term effects of daily application of Fer and SnF₂. Further optimization of the concentration of Fer, SnF₂, and H₂O₂ can be required for clinical translation and product development. Nevertheless, our data reveal that Fer and SnF₂ potentiate the therapeutic activity against tooth decay through unexpected synergistic mechanisms that target both the biological (biofilm) and physicochemical (acid enamel demineralization) traits. This simple yet effective combination therapy with the potential of fluoride delivery could advance currently available anticaries treatment while also leading to the development of ROS-based modalities for other biofilm-related diseases.

The search for new modalities encompasses novel compounds, where further development involves a long and costly process and regulatory approval. The findings that an off-the-shelf iron oxide nanoparticle formulation has a potent topical effect at a fraction (510× less) of the approved dosage together with low doses of commonly used fluoride agent can facilitate its path to clinical translation. It is noteworthy that patients with severe childhood tooth decay are often linked with iron deficiency anemia. The possibility that two major global health problems, i.e., childhood tooth decay and anemia, could be treated by using Fer and SnF₂ is indeed attractive. As Fer and SnF₂ have been used in children and adults, it opens an exciting opportunity to include combination therapy in clinical trials for caries prevention tailored to high-risk patients with iron-deficiency anemia.

### In Vitro Biofilm Model and Quantitative Analysis

Biofilms were formed using the saliva-coated hydroxyapatite disc (sHA) model as described elsewhere. Streptococcus mutans UA159, a proven virulent and well-characterized cariogenic pathogen, were grown in ultra-filtered (10 kDa, cutoff; Millipore, Billerica, MA) tryptone-yeast extract (UFTYE) broth at 37° C. and 5% CO₂ to mid-exponential phase. Briefly, HA discs (surface area of 2.7±0.2 cm²; Clarkson Chromatography Inc., South Williamsport, PA) were vertically suspended in 24-well plates using a custom-made wire disc holder and coated with filter-sterilized human saliva for 1 h at 37° C. Each sHA disc was inoculated with ˜2×10⁵ CFU of S. mutans per ml in UFTYE containing 1% sucrose at 37° C. and 5% CO₂. Topical treatment of Fer and SnF₂ or vehicle control was performed twice daily for 10 min at 0, 6, 19, and 29 h. The culture medium was changed twice daily (at 19 h and 29 h). At the end of the experimental period (43 h), the biofilms were placed in 2.8 ml of H₂O₂ (1%, v/v) for 5 min. After H₂O₂ exposure, the biofilms were removed and homogenized via bath sonication followed by probe sonication (at an output of 7 W for 30 s). The homogenized suspension was serially diluted and plated onto blood agar plates using an automated EddyJet Spiral Plater (IUL, SA, Barcelona, Spain). The numbers of viable cells in each biofilm were calculated by counting CFU. The remaining suspension was centrifuged at 5500 g for 10 min, the resulting cell pellets were then washed, oven-dried, and weighed. SnF₂ and NaF treatment groups were performed according to the same procedure.

To visualize the cell viability and EPS degradation, SYTO 9 (485/498 nm; Molecular Probes) was used for labeling live bacteria, and Alexa Fluor 647-dextran conjugate (647/668 nm; Molecular Probes) was used for labeling insoluble EPS. The 3D biofilm architecture was acquired using Zeiss LSM 800 with a 20× (numerical aperture=1.0) water dipping objective. The biofilms were sequentially scanned using diode lasers (488 and 640 nm), and the fluorescence emitted was collected with GaAsP or multi-alkali PMT detector (475-525 nm for SYTO9, and 645-680 nm for Alexa Fluor 647-dextran conjugates, respectively). ImageJ FIJI was used for biofilm visualization and quantification.

Characterization of Fer&SnF₂: The hydrodynamic diameter and zeta potential were measured using a Nano-ZS 90 (Malvern Instrument, Malvern, UK). ¹H NMR was conducted using Bruker DMX 500, which has a z-gradient amplifier and is equipped with 5 mm DUAL (1H/13C) z-gradient probe head.

ROS measurement using 3,3′,5,5′-tetramethylbenzidine (TMB) assay: The catalytic activity of Fer+SnF₂ was investigated by a colorimetric assay using TMB (Sigma-Aldrich) as a probe, which generates a blue color in the presence of H₂O₂ after reacting with ROS. Briefly, the stock solution of TMB was made in DMF (25 mg/ml). Fer (0.5 mg of Fe/ml) and SnF₂ (0.5 mg/ml, Sigma-Aldrich) were incubated (separately or combined) at room temperature in 0.1 M of sodium acetate buffer (pH 4.5) for 1 h. Afterward, 40 μl of the testing sample (Fer, SnF₂, or Fer+SnF₂) and 4 μl of TMB (100 μg) were added into 922 μl of 0.1 M sodium acetate buffer (pH 4.5), and absorbance was recorded at 652 nm. Then, 34 μl of H₂O₂ (1%, v/v) was added. After 10 min additional incubation in the dark, catalytic activities was monitored at 652 nm. For the control, 40 μl of the buffer solution was taken instead of the testing sample. To examine the effect of DMSO, a quencher of hydroxyl radical (·OH), various amounts of DMSO (0-10%) were used. The effect of pH on the catalytic activity of the combined treatment of Fer+SnF₂ was determined at three different pH values (4.5, 5.5, and 6.5), as described above. The comparison of the catalytic activity of Fer+NaF and Fer+SnF₂ was also conducted using the same protocol as described above, except after adding H₂O₂, the reaction mixture was incubated only for 5 min.

To probe the effect of barium fluoride (BaF₂, Sigma-Aldrich) on the catalytic activity of Fer, Fer (0.5 mg of Fe/ml) and BaF₂ (0.5 or 0.75 mg/ml) were incubated for 1 h in 0.1 M sodium acetate buffer (pH 4.5). Subsequently, 40 μl of the combined mixture of Fer (20 μg of Fe) and BaF₂ (20 or 30 μg) and 4 μl of TMB (100 μg) were mixed into 922 μl of 0.1 M sodium acetate buffer (pH 4.5). Afterward, H₂O₂ (1%) was added. Finally, the absorbance of TMB was monitored at 652 nm after 5 min of incubation. In a similar way, the catalytic activity of SnCl₂ (final concentration 20 μg/ml, Sigma-Aldrich) was also investigated. The effect of incubation time on the catalytic activity was investigated after incubating Fer and SnF₂ for a predetermined time.

Investigation of ROS generation using o-phenylenediamine (OPD): The enhancement of the catalytic activity of Fer in the presence of SnF₂ was further verified by employing OPD (Sigma-Aldrich) as a ROS tracking agent. Briefly, the stock solution of the combination of Fer (0.5 mg of Fe/ml) and SnF₂ (0.5 mg/ml) was incubated for 1 h in 0.1 M sodium acetate buffer (pH 4.5) at room temperature. Afterward, 40 μl of the mixture of Fer (20 μg of Fe) and SnF₂ (20 μg) and 4 μl of OPD (100 μg) were added into 922 μl of 0.1 M sodium acetate buffer (pH 4.5). After adding 34 μl of H₂O₂ (1%, v/v), the mixture was further incubated for 1 min, and the absorbance was subsequently recorded at 450 nm.

Comparison of hydroxyl radical (·OH) production: ·OH generated by Fer and Fer+SnF₂ in 0.1 M sodium acetate buffer (pH 4.5) was compared by PL technique using coumarin (Sigma-Aldrich) as a ·OH trapping molecule. First, stock solution of Fer (0.5 mg of Fe/ml) with or without SnF₂ (0.5 mg/ml) was incubated in 0.1 M sodium acetate buffer (pH 4.5) for 1 h. Afterward, Fer (20 μg of Fe/ml) with or without SnF₂ (20 μg/ml) was mixed with coumarin (0.1 mM) in a 10 mm path length cuvette, and then H₂O₂ (1%, v/v) was added to the reaction mixture to initiate the reaction. The PL intensity was recorded at 452 nm at different incubation times with an excitation wavelength of 332 nm. For the control, all the experimental conditions were the same, except that the testing samples were not used.

Iron release and catalytic activity of released iron ions: The release of iron ions from the combination of Fer and SnF₂ was investigated using inductively coupled plasma optical emission spectroscopy (ICP-OES). Briefly, 10 ml of Fer (0.5 mg of Fe/ml) was incubated with or without SnF₂ (0.5 mg/ml) for 1 h in 0.1 M sodium acetate buffer (pH 4.5). Afterward, free iron ions and intact nanoparticles were separated by centrifugation using ultrafiltration tubes (3 kDa, MWCO). The pellet was then resuspended in the same volume using 0.1 M sodium acetate buffer (pH 4.5). Finally, the iron content in the filtrate and the nanoparticle pellet was determined by ICP-OES (Spectro Genesis ICP). Additionally, the catalytic activity of the released iron and nanoparticle pellet was investigated at pH 4.5 via TMB assay. The iron release and catalytic activity of the released iron ions at pH 5.5 and 6.5 were also investigated, as discussed above.

In vivo efficacy of Fer in combination with SnF₂: In vivo efficacy was assessed using a well-established rodent model of dental caries, as reported previously. In brief, 15 days-old specific pathogen-free Sprague-Dawley rat pups were purchased with their dams from Harlan Laboratories (Madison, WI, USA). Upon arrival, animals were screened for S. mutans and were determined if they were infected with the pathogen through plating oral swabs on mitis salivarius agar plus bacitracin (MSB). Then, the animals were orally infected with S. mutans UA159, and their infections were confirmed at 21 days via oral swabbing. To simulate a clinical scenario, a topical treatment regimen was used that consisted of a short time exposure (30 s) of the agent, followed by another short time exposure (30 s) of H₂O₂ (or buffer). All infected pups were randomly placed into five treatment groups, and their teeth were treated twice daily. The treatment groups included: (1) control (0.1 M sodium acetate buffer, pH 4.5), (2) Fer only (1 mg of Fe/ml), (3) SnF₂ only (250 ppm of F), (4) ¼ Fer+¼SnF₂ (0.25 mg of Fe/ml and 62.5 ppm of F) and (5) Fer+SnF₂ (1 mg of Fe/ml and 250 ppm of F). Each group was provided the National Institutes of Health cariogenic diet 2000 (TestDiet, St. Louis, MO) and 5% sucrose water ad libitum. The experiment proceeded for 5 weeks, and their physical appearance was recorded daily. At the end of the experimental period, the jaws were surgically removed and aseptically dissected, followed by sonication to recover total oral microbiota. All of the jaws were defleshed, and the teeth were prepared for caries scoring based on Larson's modification of Keyes' system. Determination of the caries score of the jaws was performed by a calibrated examiner who was blind for the study by using codified samples. Moreover, the gingival tissues were collected for H&E staining for histopathological analysis by an oral pathologist at Penn Oral Pathology. This research was reviewed and approved by the University of Pennsylvania Institutional Animal Care and Use Committee (IACUC #805529).

16S rRNA sequencing: Cells were pelleted from dental plaque by centrifuging at maximum speed for 5 min. DNA was extracted from the pellets using the Qiagen DNeasy PowerSoil htp kit according to the manufacturer's instructions within a sterile class II laminar flow hood. Mock washes and mock extractions were included to control for microbial DNA contamination arising through the sonication and extraction processes, respectively.

PCR amplification of the V1-V2 region of the 16S rRNA gene was performed using Golay-barcoded universal primers 27F and 338R. Four replicate PCR reactions were performed for each sample using Q5 Hot Start High Fidelity DNA Polymerase (New England BioLabs). Each PCR reaction contained: 4.3 μl microbial DNA-free water, 5 μl 5× buffer, 0.5 μl dNTPs (10 mM), 0.17 μl Q5 Hot Start Polymerase, 6.25 μl each primer (2 μM), and 2.5 μl DNA. PCR reactions with no added template or synthetic DNAs were performed as negative and positive controls, respectively. PCR amplification was done on a Mastercycler Nexus Gradient (Eppendorf) using the following conditions: DNA denaturation at 98° C. for 1 min, then 20 cycles of denaturation at 98° C. for 10 sec, annealing 56° C. for 20 sec and extension 72° C. for 20 sec, last extension was at 72° C. for 8 min. PCR replicates were pooled and then purified using a 1:1 ratio of Agencourt AMPure XP beads (Beckman Coulter, Indianapolis, IN), following the manufacturer's protocol. The final library was prepared by pooling 10 μg of amplified DNA per sample. Those that did not arrive at the DNA concentration threshold (e.g., negative control samples) were incorporated into the final pool by adding 12 μl. The library was sequenced to obtain 2×250 bp paired-end reads using the MiSeq Illumina.

To analyze 16S rRNA gene sequences, QIIME2 v19.4 was used. Taxonomic assignments were obtained based on GreenGenes 16S rRNA database v.13_8 and ASV analysis of shared and unique bacterial taxa through DADA2. PCoA was performed using the library ape for R programming language. To test the differences between communities, library vegan and Unifrac distances (https://CRAN.R-project.org/package=vegan) were used. R environment (version 4.0.3) was used for statistical analysis. Non-parametrical test Wilcoxon Rank Sum Test was performed for the pairwise comparison between treatment groups for richness and Shannon diversity analysis. PERMANOVA analysis was performed for weighted Unifrac principal coordinate analysis to evaluate the differences between treatment groups. Statistical significance was considered <0.05.

Statistical analysis: The data presented as the mean±standard deviation were performed at least three times independently. One-way analysis of variance (ANOVA) followed by the Tukey test was used to determine the statistical significance between the control and the experimental groups unless otherwise stated. p values <0.05 were considered statistically significant.

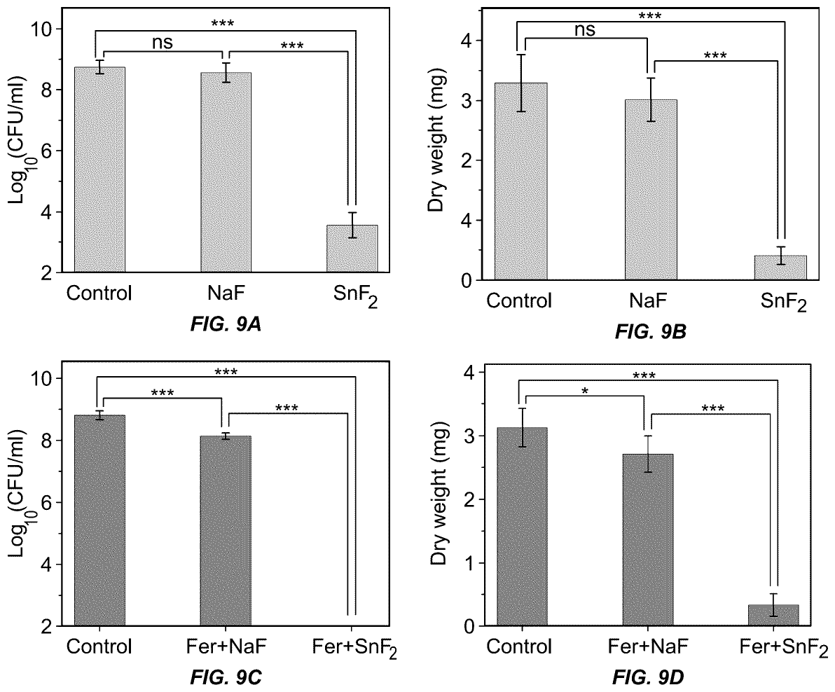

FIGS. 9-17 include bacterial viability and mass of biofilm after treatment with NaF and SnF₂, bacterial viability and mass of biofilm after treatment with Fer+NaF and Fer+SnF₂, TEM of Fer and Fer+SnF₂ after 1 h incubation, UV-visible absorption spectra of SnF₂ with or without citric acid and L-ascorbic acid, the effect of DMSO on the catalytic activity of the combined treatment of Fer and SnF₂, effect of incubation time on the catalytic activity of the combination of Fer and SnF₂, comparison of the decolorization efficiency of Fer with or without SnF₂, the plot of PL intensity of 7-hydroxycoumarion at 452 nm as a function of time with or without SnF₂, investigation of the amount of iron in the filtrate of the combined treatment of Fer and SnF₂ after 1 h incubation, and the bacterial viability and biofilm mass with the varied concentration of Fer and SnF₂.

FIGS. 9A-9B show (9A) the bacterial viability and (9B) the mass of biofilm after treatment with NaF or SnF₂ at 1000 ppm of F. FIGS. 9C-9D show (9C) the bacterial viability and (9D) the mass of biofilm after treatment with Fer+NaF or Fer+SnF₂ at 1 mg of Fe/ml, 1000 ppm of F, and 1% of H₂O₂. The data are presented with statistic symbols: *p<0.05, ***p<0.001; ns, nonsignificant; one-way ANOVA followed by Tukey test. SnF₂ can significantly inhibit the growth of S. mutans (cariogenic pathogen) and reduce the biomass when compared with NaF, indicating that SnF₂ is a much more effective biofilm treatment. When NaF or SnF₂ is combined with Fer (e.g., 1 mg of Fe/ml, an effective antibiofilm concentration) in the presence of 1% H₂O₂, the data show that the combination of Fer with SnF₂ has significantly greater antibacterial activity and reduces the biofilm biomass (dry-weight) more effectively than either alone or the combination of Fer and NaF.

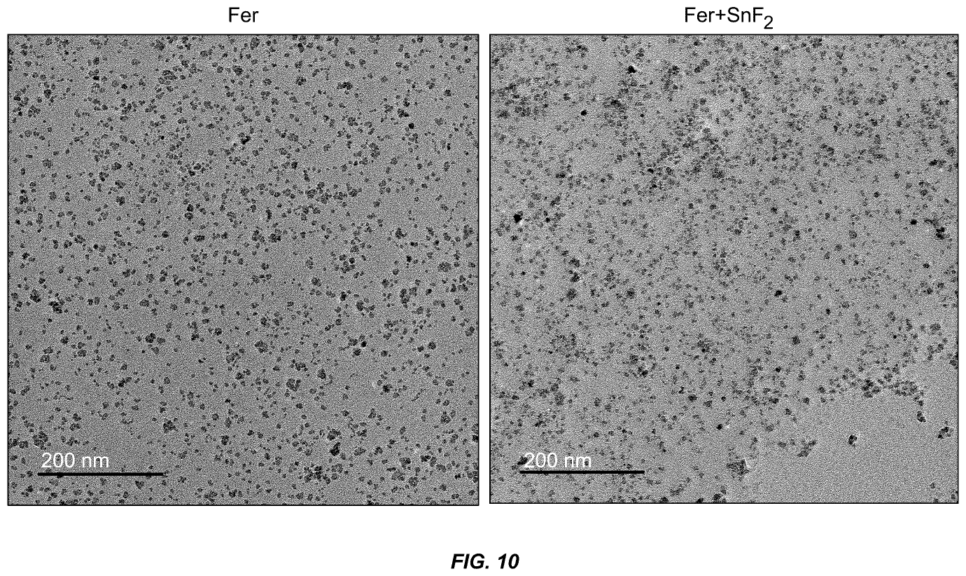

FIG. 10 shows representative TEM images of Fer and Fer+SnF₂ after 1 h incubation in 0.1 M sodium acetate buffer (pH 4.5). FIG. 10 shows that mixing Fer with SnF₂ did not affect the size.

FIG. 11 shows UV-visible absorption spectra of SnF₂ (250 ppm of F) in 0.1 M sodium acetate buffer (pH 4.5) with or without (11A) citric acid (1 mg/ml) and (11B) L-ascorbic acid (1 mg/ml) at the time points indicated. FIGS. 2d-f and 11 shows that dextran did not enhance the stability of SnF₂, whereas each material that contains carboxylic acid groups did enhance stability.

FIG. 12 shows the effects of DMSO (a quencher of ·OH) on the catalytic activity of Fer (20 μg of Fe/ml)+SnF₂ (20 μg/ml) in 0.1 M sodium acetate buffer (pH 4.5). The decrease in absorption at 652 nm shows that Fer+SnF₂ can produce ·OH. The data are presented as mean±std. The data are presented with statistic symbols: **p<0.01, ***p<0.001; ns, nonsignificant; one-way ANOVA followed by the Tukey test. Adding various amounts of DMSO (a well-known quencher of ·OH) to the mixture of Fer+SnF₂ resulted in reduced absorption of TMB at 652 nm.

FIGS. 13A-13B show the effects of incubation time on the catalytic activity of Fer (20 μg of Fe/ml) with or without SnF₂ (20 μg/ml) in 0.1 M sodium acetate buffer (pH 4.5). The increase in absorption at 652 nm shows ROS production. The data are presented as mean±std. The data are presented with statistic symbols: *p<0.05, ***p<0.001; ns, nonsignificant; one-way ANOVA followed by Tukey test. Significant amounts of ROS can be detected within 10 min incubation, gradually increasing to reach the highest level at 6 h, indicating the importance of the incubation time while mixing Fer and SnF₂.

FIGS. 14A-14B show (14A) a comparison of the decolorization efficiency of Fer+H₂O₂ with or without SnF₂. The data are presented as mean±std. The data are presented with statistic symbols: ***p<0.001; one-way ANOVA followed by the Tukey test. FIG. 14B shows representative UV-vis absorption spectra of methylene blue in the presence of Fer+H₂O₂ with or without SnF₂. Inset: Physical color of methylene blue in different conditions (left: control, middle: Fer+H₂O₂, and right: Fer+SnF₂₊H₂O₂). Fer+SnF₂ was 4-fold more effective in decoloring methylene blue than Fer.

FIG. 15 shows the change in PL intensity of 7-hydroxycoumarin at 452 nm as a function of time with or without SnF₂ (20 μg/ml). The data are presented as mean±std. SnF₂ alone did not produce a noticeable amount of ·OH.

FIG. 16 shows the amount of iron in the filtrate of the combination of Fer (0.5 mg of Fe/ml) and SnF₂ (0.5 mg/ml) after 1 h incubation at three different pH values via ICP-OES. The data are presented as mean±std. The amount of leached irons from Fer+SnF₂ formulation at circumneutral pH was low.

FIGS. 17A-17B show the bacterial viability (17A) and biofilm mass (17B) with the varied concentration of Fer (0-1 mg of Fe/ml) and SnF₂ (0-250 ppm of F). The data are presented as mean±std. ***p<0.001; one-way ANOVA followed by Tukey test. The lower amounts were capable of significantly killing the bacteria and reducing biomass compared to the control group.

### Example 2: Enhanced Stability and Catalytic Activity of Fer/SnF₂ Formulation

### SnF₂ can Enhance the Catalytic Activity of CMD-Coated Iron Oxide Nanoparticles (IONP)

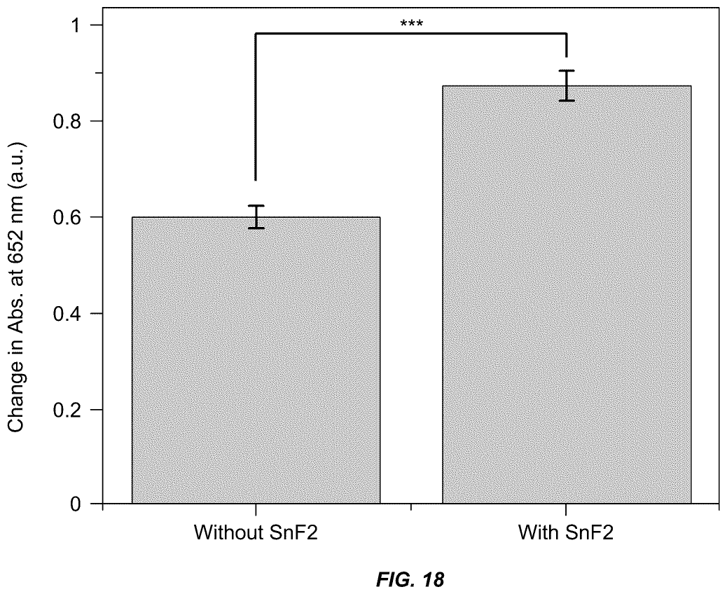

FIG. 18 provides a graph showing the comparison of the catalytic activity of CMD-coated IONP with or without SnF₂ (***p<0.001; one-way ANOVA followed by Tukey test). In agreement with the results of Fer, the catalytic activity of carboxymethyl-dextran (CMD)-coated IONP (i.e., Fer formulation contains CMD; CMD is a coating agent in Fer), increases when exposed to SnF₂. This shows that CMD can be the primary contributor to the enhanced catalytic activity of Fer in the presence of SnF₂.

### Snf₂ Enhances the Catalytic Activity of Citric Acid-Coated IONP

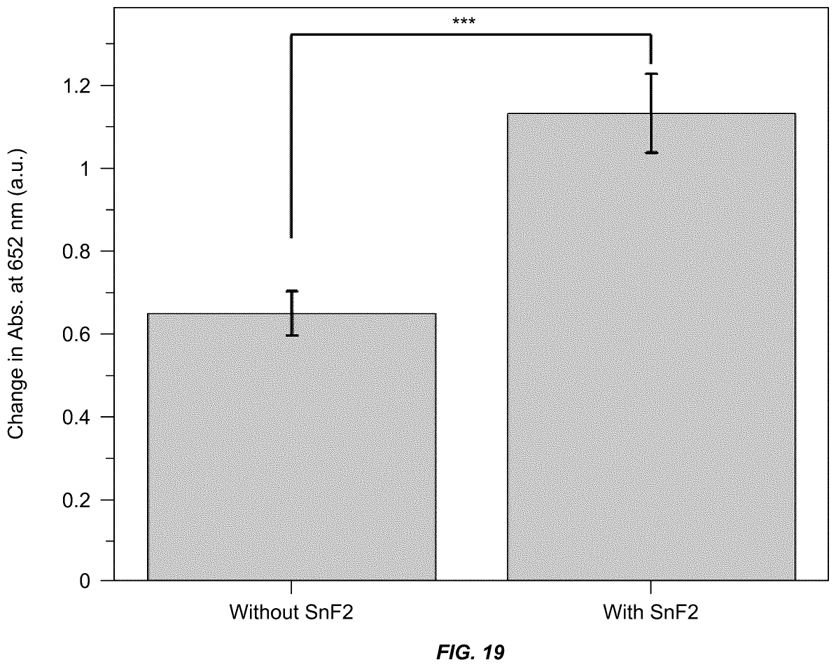

FIG. 19 provides a graph showing the comparison of the catalytic activity of citric acid-coated IONP with or without SnF₂. The increase in absorbance at 652 nm in the presence of SnF₂ indicates that SnF₂ can enhance the catalytic activity of citric acid-coated IONP (***p<0.001; one-way ANOVA followed by Tukey test). Similar to the CMD-coated IONP, a significant increase was observed in the catalytic activity of citric acid-coated IONP (citric acid contains carboxylate groups) when combined with SnF₂, suggesting that the presence of carboxylate groups, whether in CMD or citric acid coatings, can play an important role in the enhanced catalytic activity of the IONP—SnF₂ system.

### Catalytic Activity of Fer Increases in the Presence of SnF₂ if they are Pre-Mixed and Incubated

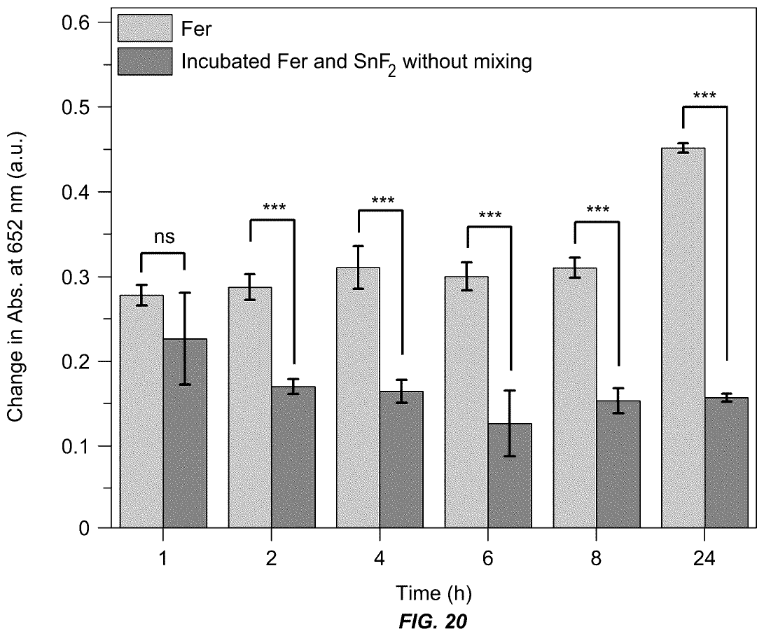

FIG. 20 provides a graph showing the effects of post-mixed SnF₂ on the catalytic activity of Fer. Post-mixed SnF₂ reduced the catalytic activity of Fer (***p<0.001; ns, non-significant; one-way ANOVA followed by Tukey test). Post-mixed SnF₂ decreases the catalytic activity of Fer, which can be due to the blocking of active sites of Fer owing to the instability of SnF₂ in aqueous solutions. This result shows the importance of pre-mixing on the Fer/SnF₂ formulation.

### Citric Acid Further Enhances the Stability of Fer/SnF₂ Formulation

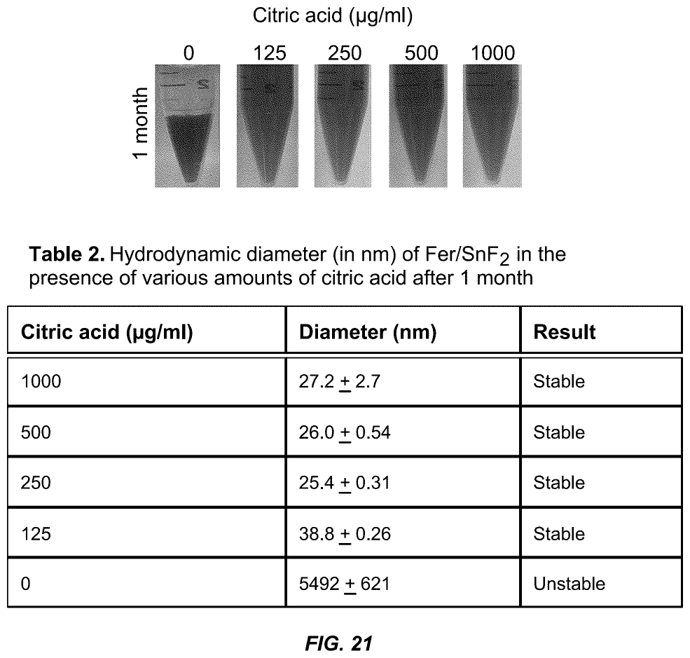

FIG. 21 provides photographs of Fer+SnF₂ when mixed with various amounts of citric acid after 1 month incubation at room temperature. The photographs show that citric acid further enhances the stability of Fer/SnF₂ formulation (no precipitation means stable) even at room temperature for a prolonged period.

TABLE 2
Hydrodynamic diameter (in nm) of Fer/SnF₂ in the
presence of various amounts of citric acid
after 1 month incubation at room temperature.

| Citric acid (μg/ml) | Diameter (nm) | Result |
|---|---|---|
| 1000 | 27.2 ± 2.7 | Stable |
| 500 | 26.0 ± 0.54 | Stable |
| 250 | 25.4 ± 0.31 | Stable |
| 125 | 38.8 ± 0.26 | Stable |
| 0 | 5492 ± 621 | Unstable |

Table 2 shows the hydrodynamic diameter of Fer/SnF₂ in the presence of various amounts of citric acid at room temperature after prolonged period (after 1 month). The size of Fer when mixed with SnF₂ at day 0 (without citric acid) is (26.0±5.3) nm. The size of Fer/SnF₂ did not change appreciably when mixed with citric acid, thereby demonstrating enhanced stability. However, the size increased remarkably in the absence of citric acid. The increased size in the absence of citric acid is attributed to reduced stability of the Fer/SnF₂ formulation after prolong time.

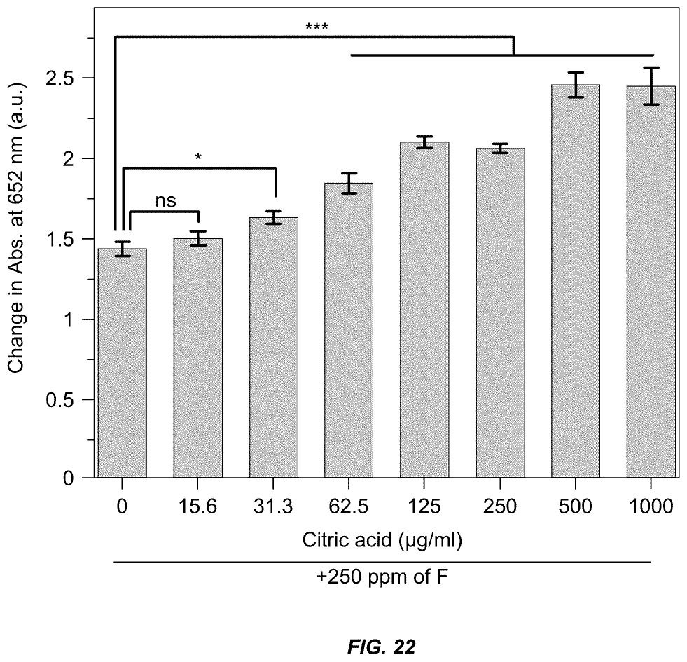

FIG. 22 provides a graph showing the evaluation of catalytic activity of Fer/SnF₂ formulation in the presence of various amounts of citric acid. After adding citric acid, the catalytic activity of Fer increases in a dose-dependent manner (ns, nonsignificant; *p<0.05, ***p<0.001; one-way ANOVA followed by Tukey test). Citric acid not only improves the stability with as little as 125 μg/ml, but also enhances the catalytic activity of the Fer/SnF₂ formulation by as much as 75%.

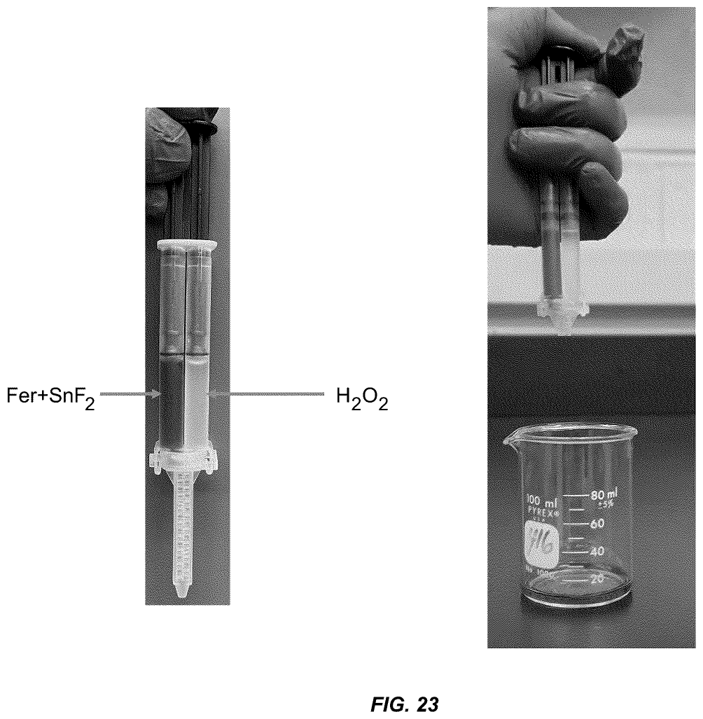

FIG. 23 shows an example dual-compartment applicator that can be used to mix and deliver Fer/SnF₂ and H₂O₂ in real time. One barrel of the syringe contains Fer/SnF₂, and other barrel contains H₂O₂. Users can mix the solutions in real-time prior to application with little to no effort.

The present disclosure is well adapted to attain the ends and advantages mentioned as well as those that are inherent therein. The particular embodiments disclosed above are illustrative only, as the present disclosure can be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above can be altered or modified, and all such variations are considered within the scope and spirit of the present disclosure.

Various publications, patents and patent applications are cited herein, the contents of which are hereby incorporated by reference in their entirety.

1. A composition for preventing and/or treating of an oral disease comprising:
(a) one or more iron oxide nanoparticles; and
(b) stannous fluoride (SnF₂).

2. The composition of claim 1, wherein the oral disease comprises dental caries.

3. The composition of claim 1, wherein the one or more iron oxide nanoparticles comprise nanoparticles having a diameter of about 1 nm to about 1000 nm.

4. The composition of claim 1, wherein the composition further comprises fluoride, copper, calcium phosphate or a combination thereof.

5. The composition of claim 1, wherein the composition further includes an active agent, wherein the active agent can include an antimicrobial, an antibiotic, or a combination thereof.

6. The composition of claim 1, wherein the one or more iron oxide nanoparticles comprise nanoparticles that have a polymeric coating.

7. The composition of claim 1, wherein the polymeric coating comprises a biopolymer, dextran, carboxymethyl dextran, chitosan, citrate, or combinations thereof.

8. The composition of claim 1, wherein the one or more iron oxide nanoparticles comprise nanoparticles that do not have a polymeric coating.

9. The composition of claim 1, wherein Sn²⁺ of the SnF₂ is bound by carboxylate groups in a carboxymethyl-dextran coating of the one or more iron oxide nanoparticles.

10. The composition of claim 1, wherein the composition is formulated as a solution, a cream, a gel, a paste, a paste, a strip, a lozenge, a gum, a gummy, a gummy bear, a resin, a sealant, a coating or a combination thereof.

11. The composition of claim 1, wherein the composition is formulated as an aqueous solution.

12. The composition of claim 1, wherein a concentration of the one or more iron oxide nanoparticles is from about 1 mg/ml to about 5 mg/ml.

13. The composition of claim 1, wherein a concentration of the SnF₂ ranges from about 1 ppm of F to about 1000 ppm of F.

14. The composition of claim 1, further comprising citric acid.

15. The composition of claim 14, wherein the iron oxide nanoparticles are coated with the citric acid.

16. The composition of claim 14, wherein the citric acid ranges from about 1 μg/ml to about 1000 μg/ml.

17. A method of preventing and/or treating a biofilm-associated disease comprising administering to a subject an effective amount of a composition comprising one or more iron oxide nanoparticles and stannous fluoride (SnF₂).

18. The method of claim 17, further comprises incubating the one or more iron oxide and SnF₂ for a predetermined time.

19. The method of claim 18, wherein the predetermined time ranges from about 1 minute to about 12 months.

20. The method of claim 17, further comprises performing a polymeric coating on the one or more iron oxide nanoparticles.

21. The method of claim 20, wherein the polymeric coating comprises a biopolymer, dextran, carboxymethyl dextran, chitosan or combinations thereof.

22. The method of claim 17, wherein the biofilm is generated by a biofilm-forming microbe, wherein the biofilm-forming microbe is selected from the group consisting of S. mutans, P. aeruginosas, E. coli, E faecalis, B. subtilis, S. aureus, Vibrio cholerae, Candida albicans and a combination thereof.

23. The method of claim 17, further comprising creating a protective layer at a target surface using the composition, wherein the protective layer is an antibacterial layer that is enriched with Sn, iron, and fluoride for preventing enamel demineralization.

24. The method of claim 17, wherein the presence of the biofilm is present on a surface of a tooth, an industrial material, a naval material, skin, mucosal/soft tissue, an interior of a tooth (endodontic canal), lung (cystic fibrosis), urinary tract or a medical device.

25. The method of claim 14, wherein the composition comprises citric acid.

26. The method of claim 14, wherein the one or more iron oxide nanoparticles are coated with the citric acid.

27. The method of 25, wherein the citric acid ranges from about 1 μg/ml to about 1000 μg/ml.
